(19) United States
(12) Patent Application Publication
(10) Pub. No.: US 2022/0126066 A1
(43) Pub. Date: Apr. 28, 2022

(54) FORCE AND LOAD SENSOR AND MONITORING DEVICE FOR DEFINING INJURY DURING URETERAL ACCESS SHEATH DEPLOYMENT

(71) Applicant: THE REGENTS OF THE UNIVERSITY OF CALIFORNIA, Oakland, CA (US)

(72) Inventors: Ralph V. Clayman, Orange, CA (US); Kamaljot S. Kaler, Orange, CA (US); Jamie Landman, Orange, CA (US); Michael John Klopfer, Irvine, CA (US); Guann-Pyng Li, Irvine, CA (US)

(21) Appl. No.: 17/570,776

(22) Filed: Jan. 7, 2022

Related U.S. Application Data
(63) Continuation-in-part of application No. 15/963,851, filed on Apr. 26, 2018, now abandoned.
(60) Provisional application No. 62/491,011, filed on Apr. 27, 2017.

(51) Int. Cl. A61M 25/06; A61B 90/00; A61M 25/01; A61M 25/00

(52) U.S. Cl. CPC A61M 25/0668; A61B 90/06; A61M 2025/0681; A61M 25/0017; A61B 2090/064; A61M 25/0105

(57) ABSTRACT

Various embodiments herein comprise a handheld load sensing device. In one embodiment, the device comprises a force sensor (e.g., a mechanical spring or an electromechanical load cell) adapted to receive a force, an input/output interface, and an adjustable attachment mechanism adapted to reversibly attach a surgical device. In another embodiment, the force sensor detects the amount of force applied during the deployment of the surgical device in a patient and outputs a force value representative thereof via the input/output interface. Also provided herein are methods of making and using load sensing devices.

### CROSS-REFERENCES TO RELATED APPLICATIONS

This application is a continuation-in-part and claims benefit of U.S. patent application Ser. No. 15/963,851 filed Apr. 26, 2018, which is a non-provisional application and claims benefit of U.S. Provisional Application No. 62/491,011 filed Apr. 27, 2017, the specifications of which are incorporated herein in their entirety by reference.

### FIELD OF THE INVENTION

The present disclosure relates to the field of medical devices, specifically surgical devices.

### BACKGROUND OF THE INVENTION

The ureteral access sheath (UAS) was first introduced in the 1970's in order to provide an accessible conduit to the kidney for rapid and safe repeated introduction and removal of a rigid or flexible ureteroscope. Today, UAS is commonly used during flexible ureteroscopy (URS) for stone management and to diagnose and treat other upper urinary tract diseases, such as strictures or upper urinary tract tumors. However, UAS is not universally embraced due to fear of ureteral injury, which may result from the excessive force being applied during UAS placement.

Currently, there is no device that measures force during UAS deployment; essentially, the UAS is deployed blindly, and there is a risk of injury, depending upon the operator's experience and force applied. While some devices may be present for broad force sensing, and while surgical robotic systems may have used force feedback with regard to instruments abutting tissue, there remains a need for a direct handheld system available to determine force applied during the manual passage of a catheter and/or sheath deployment.

Using a previously author-designed unique electromechanical device, the Inventors determined that a force of 3 Newtons or less induces little change in the ureter and thus the urologist, at the end of a ureteroscopic procedure would likely not need to leave an indwelling stent, thereby reducing the cost of the procedure and postoperative discomfort for the patient. Additionally, the authors determined that the porcine and the human ureter can tolerate a force up to 6 Newtons without causing splitting of the ureter; however, at this higher force, there is some ureteral edema which would necessitate placement of an indwelling stent. The threshold for splitting the ureter begins at 8 Newtons. Using this information, the Inventors have developed a far simpler force sensor than their original device to alert the urologist to key levels of force during the passage of the ureteral access sheath (i.e., at 3, 6, and 8 Newtons).

### BRIEF SUMMARY OF THE INVENTION

It is an objective of the present invention to provide systems and devices that allow for measurement of force applied to an insertion sheath during urologic procedures providing indication to the user about thresholds for dangerous forces, as specified in the independent claims. Embodiments of the invention are given in the dependent claims. Embodiments of the present invention can be freely combined with each other if they are not mutually exclusive.

In some aspects, the invention disclosed is a simplified force measurement device for catheter and sheath insertion procedures that alerts the user (e.g., a urologist) to critical levels of force during the passage of the ureteral access sheath (e.g., at 3, 6, and 8 Newtons or just at 6 N. Currently, the device of the present invention has been tested and directed towards force measurement during urologic procedures for urolithiasis in which a ureteral access sheath is inserted; however, the device may be utilized on a broader level when passing any catheter, needle, or other devices into the human body or when retracting tissues that may be delicate (e.g., veins or nerves).

In some embodiments, the present invention may feature a handheld load sensing device comprising a cylindrical outer casing, a plunger slidably coupled to the cylindrical outer casing, and a spring disposed within the exterior lumen of the cylindrical outer casing. In some embodiments, the cylindrical outer casing comprises a first casing end, a second casing end, an exterior lumen, and a guidewire lumen disposed within the exterior lumen. In some embodiments, the first casing end comprises a first opening, and the second casing end comprises a second opening for accessing the guidewire lumen. In some embodiments, the plunger comprises a first plunger end and a second plunger end having a handle disposed thereon. In some embodiments, the first plunger end is disposed through the first opening of the first casing end and within the exterior lumen. In some embodiments, the first plunger end comprises a plunger lumen. In some embodiments, the plunger lumen, and the guidewire lumen of the cylindrical outer casing are fluidly coupled. In some embodiments, a first spring end of the spring rests against the second casing end and a second spring end rests against the first plunger end. In some embodiments, the guidewire lumen is disposed through the spring such that the spring wraps around the guidewire lumen. In some embodiments, when the handle of the plunger is pushed towards the first casing end, the plunger pushes against and compresses the spring. In some embodiments, the compression of the spring corresponds to an applied force. In other embodiments, the displacement of the spring corresponds to an applied force.

In other embodiments, the present invention may further feature a handheld load sensing device, comprising a force sensor to receive a force, an input/output interface, and an adjustable attachment mechanism adapted to reversibly attach to a surgical device which is passed over a guidewire. In some embodiments, the force sensor detects the amount of force applied to the butt end of a catheter during deployment of the surgical device over the guidewire in a patient and outputs a force value representative thereof through the input/output interface.

One of the unique and inventive technical features of the present invention is the real-time force readings. In earlier electromechanical devices developed by the Inventors, the force sensor involved real-time force readings via a Bluetooth™ interface with associated software capable of measuring force in hundredths of a Newton. In this iteration, a simpler, mechanical force sensor device provides real-time feedback to the user at predefined force thresholds (e.g., at three predefined thresholds at 3 N, 6 N, and 8 N or at a single predefined threshold of 6 N). Without wishing to limit the invention to any theory or mechanism, it is believed that the technical feature of the present invention advantageously provides for enabling a surgeon to avoid any injury to the ureter by adhering to a 6 N threshold. Further for UAS placed at the 3 N force threshold, surgeons may be able to safely eliminate ureteral stent placement after ureteroscopy; this alone would decrease the attendant morbidity and cost of ureteroscopic stone removals. None of the presently known prior references or work has the unique inventive technical feature of the present invention.

Furthermore, the prior references teach away from the present invention. For example, prior devices do not measure the force being measured as a sheath is being inserted into a ureter to prevent damage. Additionally, prior references do not comprise a strictly mechanical way to measure force through a lumen. Moreover, none of the devices described the prior references teach of a handheld device that measures force.

Furthermore, the inventive technical feature of the present invention contributed to a surprising result. For example, the point at which a ureteral stent could be eliminated after a ureteroscopic procedure became clearly defined as 3 N. This is far superior to the “visual” inspection of the ureter after a case, during which time areas of injury may be overlooked. Failure to place a stent when the ureter has been injured could lead to pain, obstruction, and decreased function of the affected kidney.

Various embodiments disclosed herein include a load sensing device, comprising: a load cell adapted to receive a force: an input/output interface; and an adjustable attachment mechanism adapted to reversibly attach a surgical device; wherein the load cell detects the amount of applied force during the deployment of the surgical device in a patient and outputs a force value representative thereof via the input/output interface in a real time continuous mode. In one embodiment, the force monitoring and force output is done continuously. In one embodiment, the device further comprises software adapted to continuously collect and store the force value. In one embodiment, the data collection and storage is done wirelessly. In one embodiment, the data collection and storage is done by Bluetooth™ technology. In one embodiment, the adjustable attachment mechanism is a screw port. In one embodiment, the adjustable attachment mechanism is a mechanical screw port. In one embodiment, the surgical device is a device used in an endoscopic, laparoscopic, robotic, and/or minimally invasive surgical procedure. In one embodiment, the surgical device is a ureteral access sheath. In one embodiment, the input/output interface is disposable. In one embodiment, a user is capable of measuring input force during the deployment of the surgical device in a patient. In one embodiment, the device is a handheld device. In one embodiment, the device further comprises indicators (e.g., aural, visual, or tactile) to alert the user as the input force increases. In one embodiment, the indicator is a visual indicator, comprising flashing green, yellow and red lights. In one embodiment, the indicator is an audio indicator, comprising increasing sound or disharmonious sound as the force increases. In one embodiment, the indicator is tactile, providing detents over which the device would ride at different levels of force which would be perceived by the surgeon.

Various embodiments disclosed herein further include a method of making a load sensing device, comprising: (a) providing a load cell, an input/output interface, and an adjustable attachment mechanism adapted to reversibly attach a surgical device; (b) connecting the load cell to the adjustable attachment mechanism such that the load cell receives and monitors the amount of applied force during the deployment of the surgical device in a patient; and (c) connecting the load cell to the input/output interface such that the input/output interface outputs a force value representative of the amount of applied force. In one embodiment, the force monitoring and force output is done continuously. In one embodiment, the device further comprises providing a software adapted to continuously collect and store the force value. In one embodiment, the data collection and storage is done wirelessly. In one embodiment, the data collection and storage is done by Bluetooth™ technology. In one embodiment, the adjustable attachment mechanism is a screw port. In one embodiment, the adjustable attachment mechanism is a mechanical screw port. In one embodiment, the surgical device is a device used in an endoscopic, laparoscopic, robotic, and/or minimally invasive surgical procedure. In one embodiment, the surgical device is a ureteral access sheath. In one embodiment, the input/output interface is disposable. In one embodiment, the device is a handheld device. In one embodiment, the method further comprises indicators to alert the user as the input force increases. In one embodiment, the indicator is a visual indicator, comprising flashing green, yellow and red lights. In one embodiment, the indicator is an audio indicator, comprising increasing or disharmonious sound as the force increases. In one embodiment, the indicator is a detent or ratchet that provides physical tactile feedback to the user as the force increases. In one embodiment, sterile procedures are used throughout the manufacturing process.

Other embodiments disclosed herein include a method of using a load sensing device during a surgical procedure of a patient comprising: (a) providing a load sensing device comprising a load cell adapted to receive a force; an input/output interface; and an adjustable attachment mechanism adapted to reversibly attach a surgical device; (b) providing a medical device to be inserted in the patient, wherein the force sensor is not introduced at any time into the patient; (c) attaching the load sensing device to the medical device; and (d) inserting the medical device in the patient, wherein the load sensing device monitors and outputs the force value during the medical device insertion. In one embodiment, sterile procedures are used during the use process. In one embodiment, the medical device is a device used in an endoscopic, laparoscopic, robotic, and/or minimally invasive surgical procedure. In accordance with various embodiments herein, the device would be sterile as it would be used in the operating room environment. In one embodiment, the medical device is a ureteral access sheath with a built-in force sensor. In one embodiment, the load sensing device is a handheld device. In one embodiment, the load sensing device further comprises indicators to alert the user as the input force increases. In one embodiment, the indicator is a visual indicator, comprising flashing green, yellow and red lights. In one embodiment, the indicator is an audio indicator, comprising increasing and/or disharmonious sound as the force increases. In one embodiment, the indicator is a detent or ratchet that provides physical tactile feedback to the user as the force increases. In another embodiment, at no point is the load sensing device inserted in the patient. In another embodiment, information from the load cell is incorporated into a simple disposable device. In another embodiment, the simple disposable device is intrinsic to the medical device. In another embodiment, the simple disposable device is extrinsic to the medical device. In another embodiment, the user is not able to exert more than 8 N of force when passing a ureteral access sheath.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows an overview of the devices as described herein.

FIG. 2 shows a resistance locking mechanism of the devices described herein

FIGS. 3A and 3B show a pawl and ratchet release mechanism of the present invention.

FIG. 4 shows a non-limiting example of how the indicator window of the cylindrical outer casing could be utilized to show visual indicators.

FIG. 5 shows the force sensing devices described herein. The green area indicates up to 3 N, whereas the full yellow area indicates that 6 N has been reached. The part yellow, part red area indicates forces in excess of 6 N. The full red area indicates 8 N has been reached.

FIGS. 6A, 6B, 6C, 6D, and 6E shows, in accordance with embodiments herein, ureteral access sheath force sensing device and screenshot of software. FIG. 6A shows a prototype design; FIG. 6B shows a top view of the device; FIG. 6C shows a side view of the device; and FIG. 6D shows a whole device in view with <3 N applied. FIG. 6E shows a screenshot of a real-time force sensor reading as transmitted to a tablet computer via Bluetooth™.

FIG. 7 shows, in accordance with embodiments herein, force measurements of first pass ureteral dilators and UAS deployments in six right porcine ureters as measured over deployment time.

FIG. 8 shows, in accordance with embodiments herein, force measurements of first pass ureteral dilators and UAS deployments in six left porcine ureters as measured over deployment time.

FIG. 9 shows, in accordance with embodiments herein, mean application force during ureteral dilator deployment using different size dilators up to 24 F in a porcine study. Note that in 25% of the cases, an 18 F dilator could be passed, and in one porcine ureter, a 24 F dilator could be passed at only 6 N. This was done before and one week after placing an indwelling ureteral stent.

FIG. 10 shows, in accordance with embodiments herein, peak application force during ureteral dilator and access sheath deployment and the point at which splitting of the urothelium is first noted in the porcine ureter at 8 N.

FIGS. 11A, 11B, and 11C show, in accordance with embodiments herein, research results utilizing UAS devices. FIG. 11A shows a medial view of the normal urothelium of the right proximal ureter. FIG. 11B shows a medial wall injury of the right proximal porcine ureter and corresponding fluoroscopic image after 13 F UAS placement. FIG. 11C shows a lateral wall injury of the right proximal porcine ureter and corresponding fluoroscopic image after 14 F UAS placement.

FIG. 12 shows, in accordance with embodiments herein, a UAS force sensing device, including an example of a design.

FIG. 13 shows in accordance with an embodiment herein, a Luer-Lok interfacing ureteral access sheath force measurement and protection device. Pictured is the following: (1) Luer-Lock female connector to connect to the male Luer-Lok connector on the stylet on the top of the ureteral access sheath device; (2) floating shaft; (3) device body; (4) force gauge; (5) maximum force indicator; (6) exit point of the guidewire at the top of the floating shaft.

FIG. 14 shows, in accordance with an embodiment herein, a shaft-coupling embodiment of the device pictured in FIG. 13. Pictured is the following: a controlled forceps arm; (2) ureteral access sheath shaft; (3) idler forceps arm attached to the shaft of the UAS; a force gauge; and (6) release button.

FIG. 15 shows, in accordance with an embodiment herein, an integrated force measurement and safety release ureteral access sheath. Pictured is the following: (1) finger grip; (2) ureteral access sheath device body; (3) ureteral access sheath shaft (UAS); high-K spring; collapsible bellows; (6) force gauge; slide channel; magnet and ferrous ring pair.

FIG. 16 shows, in accordance with an embodiment herein, the mechanism of the device pictured in FIG. 14. Pictured is the following: Pliable roller on a shaft supported by rotational bearings—connected; (1) and (2) Pliable roller on shaft supported by rotational bearings—idler; (3) pivot; (4) force clutch—shifts coaxially as force is increased as sloped teeth un-mesh; (5) high-k spring; (6) threaded support; (7) force clutch retainer and reset button; (8) force gauge.

FIG. 17 shows, in accordance with an embodiment herein, an example of the original Ureteral Access Sheath (UAS) Force Sensor Device as used by the authors in porcine and clinical studies.

FIGS. 18A, 18B, and 18C show, in accordance with an embodiment herein, sample images of various post ureteroscopic lesion scale (i.e., PULS) scores which range from 0 (no visible injury) to 5 (complete ureteral disruption) FIG. 18A is shown in green—PULS grade 0 demonstrates no lesion (top left) and insignificant lesions as pointed out by red arrows. An insignificant mucosal hematoma (top right), an insignificant mucosal edema (bottom left), and an insignificant mucosal molding (bottom right) are shown along with a guidewire in the field of view. FIG. 18B is shown in yellow—PULS grade 1 demonstrates a superficial mucosal lesion (top left) and significant lesions as pointed out by red arrows. A mucosal hematoma (top right), a larger amount of mucosal edema (bottom left), and a mucosal molding (bottom right) are shown with a guidewire also in view. FIG. 18C is shown in red—PULS Grade 2 demonstrates submucosal lesion (top row) and PULS Grade 3 perforation with less than 50% (partial) transection (bottom row); A video image PULS grade 2 is shown in the top left of FIG. 18C and a fluoroscopy image PULS grade 2 (no extravasation of contrast media) is shown in the top right of FIG. 20C. A video image PULS grade 3 is shown in the bottom left of FIG. 18C and fluoroscopy image PULS grade 3 (extravasation of contrast media).

FIG. 19 shows, in accordance with an embodiment herein, a chart of PULS grade 0-5.

FIG. 20 shows, in accordance with an embodiment herein, an example of a force measuring device connected with the UAS. With demarcations only at 4 and 8 N.

FIGS. 21A, 21B, and 21C show several embodiments of the force sensing device described herein.

FIG. 22 shows, in accordance with an embodiment herein, a force sensing device as described.

### DETAILED DESCRIPTION OF THE INVENTION

Following is a list of elements corresponding to a particular element referred to herein:
100 Cylindrical Outer Casing
110 First Casing End
111 First Opening
120 Second Casing End
122 Second Opening
131 Exterior Lumen
132 Guidewire Lumen
200 Plunger
210 First Plunger End
220 Second Plunger End
230 Plunger Lumen
300 Handle
400 Spring
410 First Spring End
420 Second Spring End

All references, publications, and patents cited herein are incorporated by reference in their entirety as though they are fully set forth. Unless defined otherwise, technical, and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As disclosed herein, the inventors have developed a novel medical device that is useful during surgery. In one embodiment, the device may be used for assisting and training surgeons by measuring force during ureteral access sheath deployment in urological surgeries, but applicable to catheter and sheath insertion for urological, laparoscopic, robotic, and vascular procedures. In one embodiment, the device is a force measurement device for catheter and sheath insertion procedures.

A force of less than 3 N results in no visible ureteral injury (post-ureteroscopic lesion score (PULS) of 0 or 1)), which translates into a safe threshold that should not require a ureteral stent placement in uncomplicated cases as it indicates that post ureteroscopic ureteral edema is unlikely. A force of 6 N was determined to be a threshold below which no high-grade ureteral injury occurred (i.e., splitting of the urothelium—a PULS 3 or higher). At a force of 8 N or more, injury is likely at the PULS 3 level in upwards of 20% of cases.

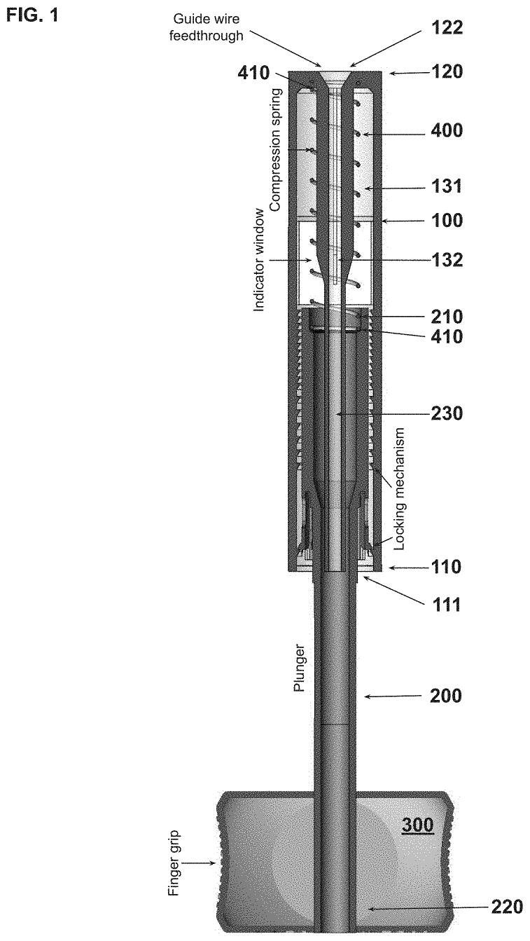

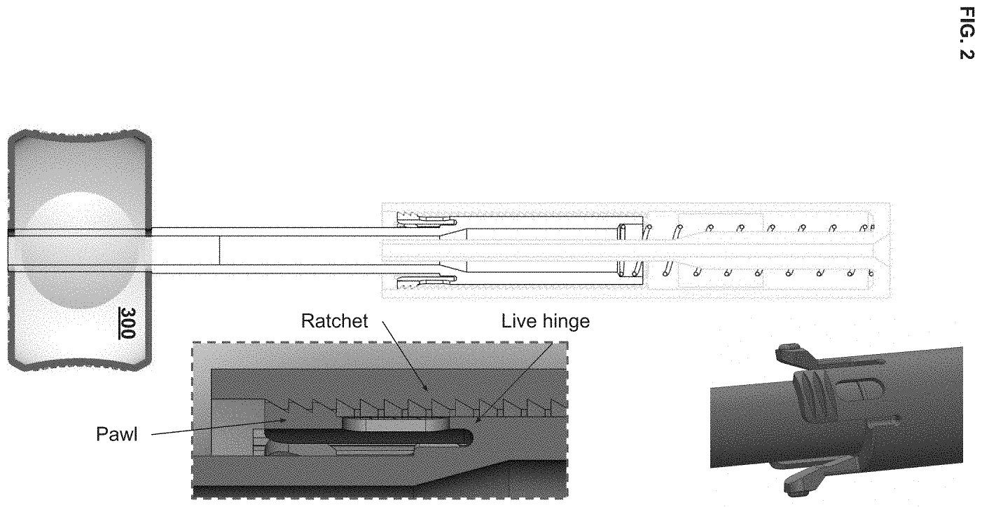

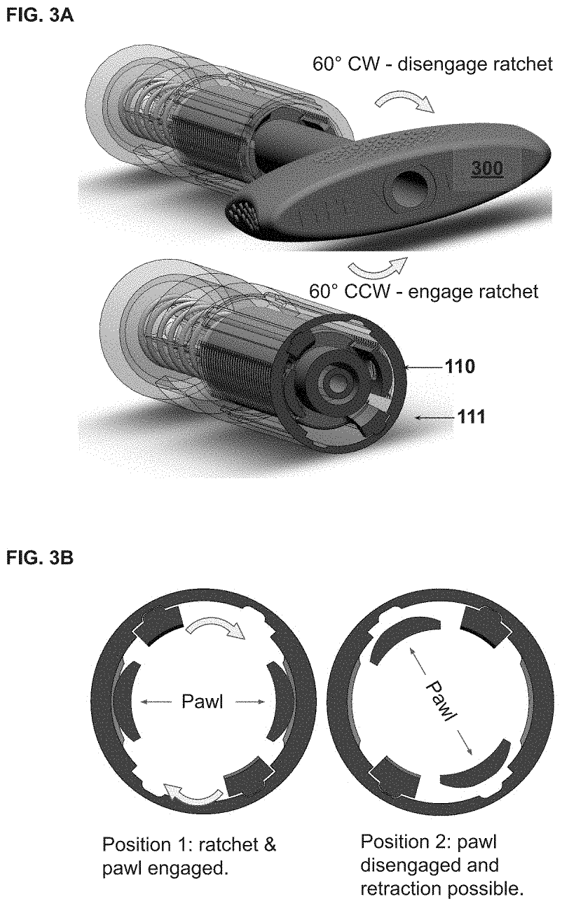

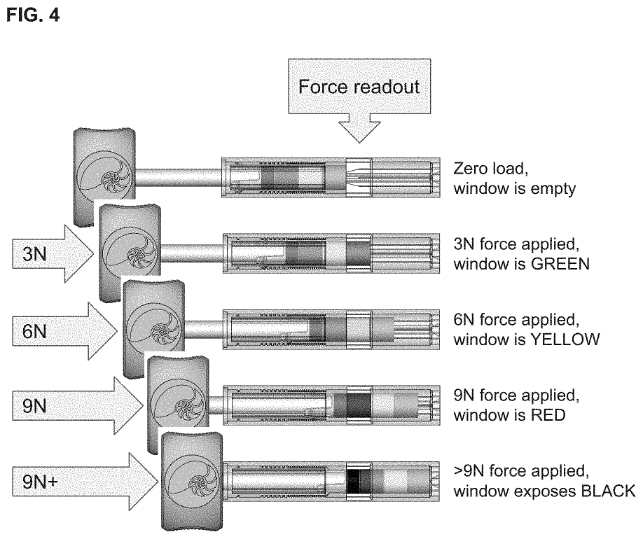

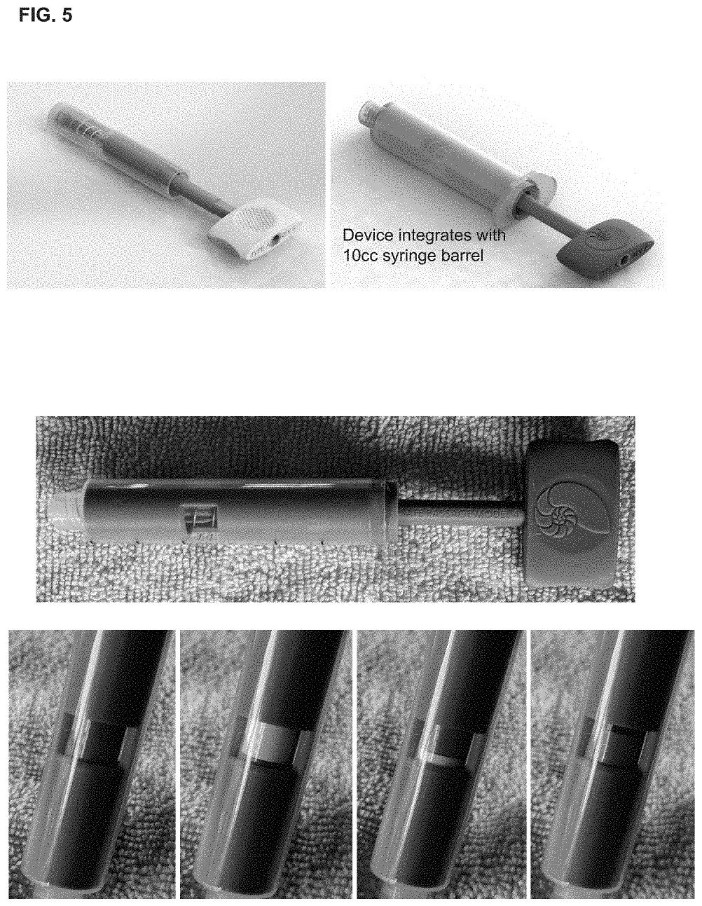

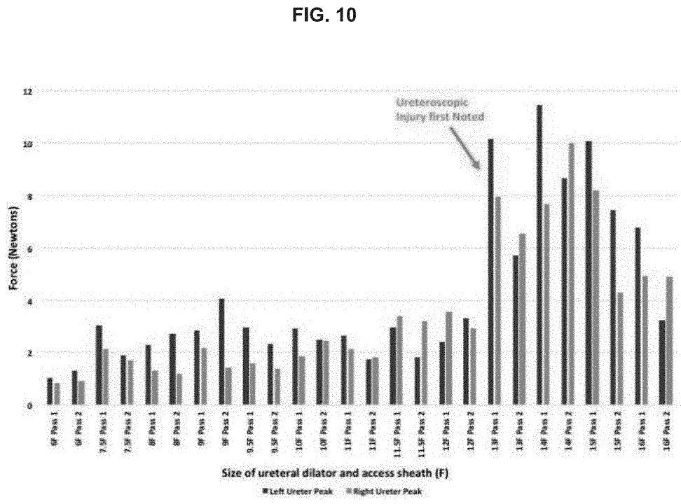

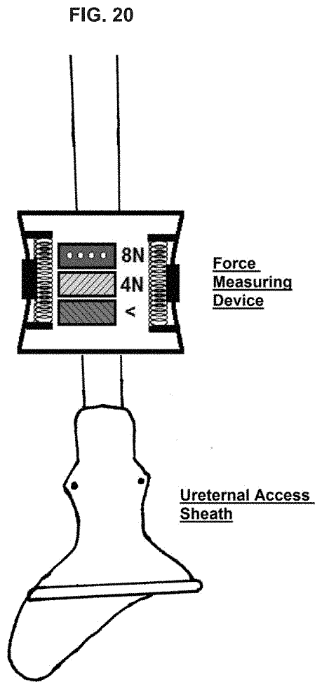

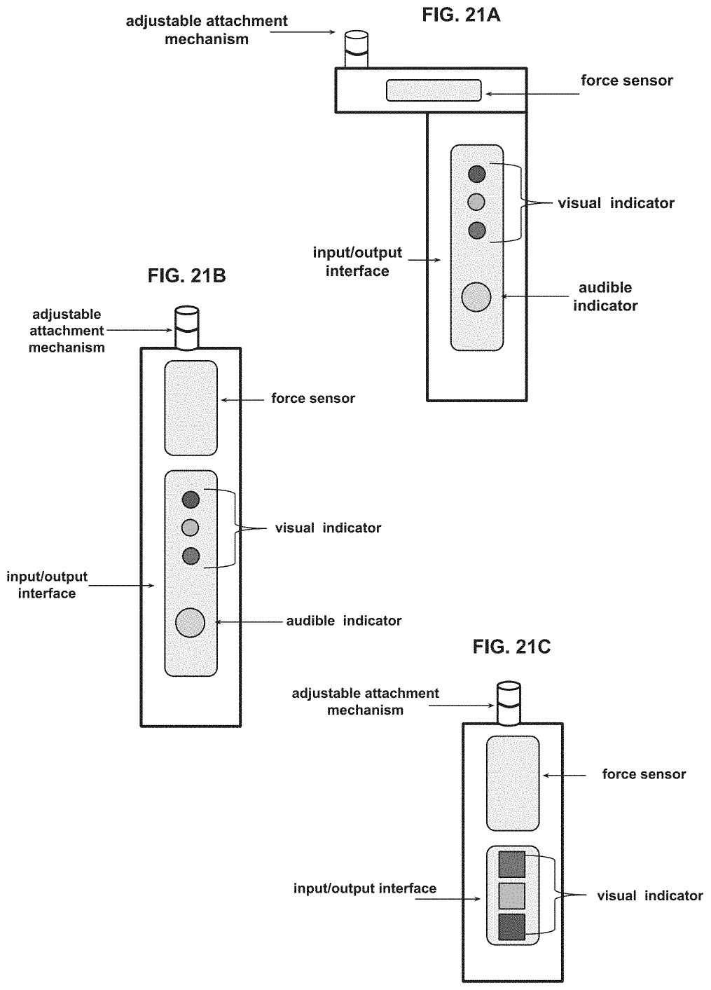

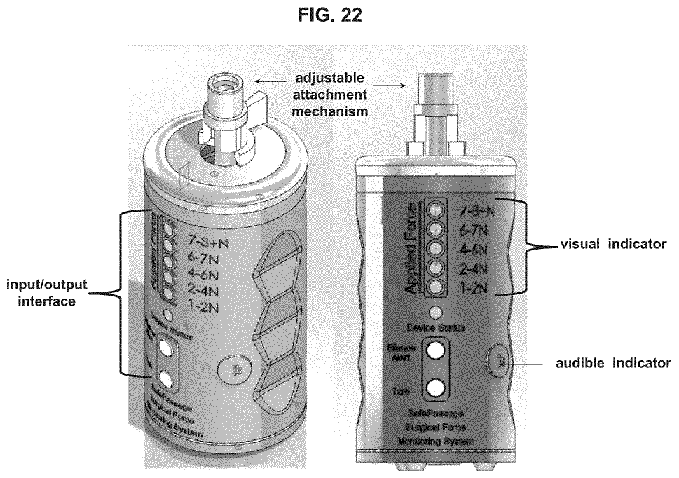

Referring now to FIGS. 1-22, the present invention features a device to measure force. In some embodiments, devices described herein could be equipped with an alarm as to when a safe force applied to a device is being approached or has been reached and should not be exceeded. This would apply to the passage of any catheter, needle, trocar, or other assembly into the tissues of the human body or during surgical dissection with regard to the amount of force being exerted on a nerve or other sensitive tissue during surgical retraction such as blood vessels.

The present invention may feature a handheld load sensing device, comprising a cylindrical outer casing (100), a plunger (200) slidably coupled to the cylindrical outer casing (100), and a spring (400) disposed within the exterior lumen (131) of the cylindrical outer casing (100). In some embodiments, the cylindrical outer casing (100) comprises a first casing end (110), a second casing end (120), an exterior lumen (131), and a guidewire lumen (132) disposed within the exterior lumen (131). In some embodiments, the first casing end (110) comprises a first opening (111) and the second casing end (120) comprises a second opening (122) for accessing the guidewire lumen (132). In some embodiments, the plunger (200) comprises a first plunger end (210) and a second plunger end (220), having a handle (300) disposed thereon. In some embodiments, the first plunger end (210) is disposed through the first opening (111) of the first casing end (110) and within the exterior lumen (131). In some embodiments, the first plunger end (210) comprises a plunger lumen (230). In some embodiments, the plunger lumen (230) and the guidewire lumen (132) of the cylindrical outer casing (100) are fluidly coupled. In some embodiments, a first spring end (410) of the spring rest against the second casing end (120) and a second spring end (420) rest against the first plunger end (210). In some embodiments, the guidewire lumen (132) is disposed through the spring (400) such that the spring (400) wraps around the guidewire lumen (132). In some embodiments, when the handle (300) of the plunger (200) is pushed towards the first casing end (110), the plunger (200) pushes against and compresses the spring (400). In some embodiments, the compression of the spring (400) corresponds to an applied force. In other embodiments, the displacement of the spring (400) corresponds to an applied force.

The present invention may further feature a handheld load sensing device, comprising a force sensor to receive a force, an input/output interface, and an adjustable attachment mechanism adapted to reversibly attach to a surgical device which is passed over a guidewire. In some embodiments, the force sensor detects the amount of force applied to the butt end of a catheter during deployment of the surgical device over the guidewire in a patient and outputs a force value representative thereof through the input/output interface.

In some embodiments, the force sensor is mechanical (e.g., a spring). In some embodiments, the force sensor is a spring. In other embodiments, the force sensor is electromechanical (e.g., a load cell). In some embodiments, the force sensor is a load cell. In some embodiments, the surgical device is a ureteral access sheath (UAS) or an endoscope. In some embodiments, the endoscope is rigid. In other embodiments, the endoscope is flexible.

In some embodiments, the devices described herein may be made of plastic or metal. In some embodiments, devices derived herein made of plastic may be side-mounted rather than end mounted. In other embodiments, devices derived herein made of plastic may be end-mounted (i.e., the device is directly in line with the UAS or catheter). In other embodiments, the devices described herein made of plastic may be side-mounted (i.e., the device is offset from the UAS or catheter). In other embodiments, devices derived herein made of metal may be end mounted. In other embodiments, the devices described herein made of metal may be side mounted.

In some embodiments, the devices described herein may undergo gas sterilization. In some embodiments, the devices described herein are single-use, disposable, or sterilizable reusable items. In some embodiments, the internal portion of the device is hollow to allow the passing of a guidewire through the device. In some embodiments, the sliding portion of the device is isolated with linear bearings providing smooth sliding motion.

In some embodiments, the devices described herein have a Luer-Lock™ style interface that connects (either locking or resting that will separate with the removal of force) to the stylet portion of a urological ureteral insertion sheath. In some embodiments, the second casing end (120) of the cylindrical outer casing (100) further comprises an interface for an access sheath (e.g., a ureteral access sheath (UAS). In other embodiments, the second casing end (120) of the cylindrical outer casing (100) further comprises an interface for an endoscope. In some embodiments, the endoscope is flexible or rigid. In some embodiments, the interface comprises a Luer-lock style interface. In some embodiments, the interface is positioned distal to the UAS point of insertion. In other embodiments, the interface is positioned distal to a point of insertion of the UAS or the endoscope. In other embodiments, the second end (120) of the cylindrical outer casing (100) abuts the end of an access sheath (e.g., a ureteral access sheath (UAS)) without locking into it. In some embodiments, a guidewire is passed through the Luer-lock style interface and into the guidewire lumen (132) of the cylindrical outer casing (100) and/or into the lumen (230) of the plunger (200).

In some embodiments, the devices described herein allow for measurement of the catheter sheath deployment/insertion force via an end mounted load cell or spring-locking mechanism. In certain embodiments, spring displacement is used as the primary method of force measurement. Displacement of 0 to maximum range (e.g., 1¼ max travel) would correspond to an applied force of 0 to 8 N. The restorative force would be provided by a linear spring with a fixed Hooke's constant (N/m) value.

“Hooke's Law” states a force needed to extend or compress a spring by some distance scales linearly with respect to that distance—that is, F=kx, where k is a constant factor characteristic of the spring (i.e., a Hooke's constant), where F is the force applied to the spring (either in the form of strain or stress) and where X is the displacement of the spring.

In certain embodiments, the springs used in accordance with the devices described herein comprise a Hooke's constant of 359 N/m. In other embodiments, the springs used in accordance with the device described herein comprise a Hooke's constant of about 200 N/m, or about 225 N/m, or about 250 N/m, or about 275 N/m, or about 300 N/m, or about 325 N/m, or about 350 N/m, or about 375 N/m, or about 400 N/m, or about 425 N/m, or about 450 N/m, or about 475 N/m, or about 500 N/m.

As used herein, a “compression spring rate” refers to the change in load per unit of deflection, expressed in pounds per inch and is determined by the amount of force, in pounds, required to constrict a spring by one inch. In certain embodiments, the spring used in accordance with the devices described herein comprises a compression spring rate of 2.051 lb/in. In other embodiments, the spring used in accordance with the devices described herein comprises a compression spring rate of about 1.00 lb/in, or about 1.25 lb/in, or about 1.50 lb/in, or about 1.75 lb/in, or about 2.00 lb/in, or about 2.25 lb/in, or about 2.50 lb/in, or about 2.75 lb/in, or about 3.00 lb/in, or about 3.25 lb/in, or about 3.50 lb/in.

In certain embodiments, the spring used in accordance with the devices described herein compresses 0.11 in/N. In some embodiments, In certain embodiments, the spring used in accordance with the devices described herein compresses about 0.10 in/N, or about 0.15 in/N, or about 0.20 in/N, or about 0.25 in/N, or about 0.30 in/N, or about 0.35 in/N, or about 0.40 in/N, or about 0.45 in/N, or about 0.50 in/N, or about 0.55 in/N, or about 0.60 in/N, or about 0.65 in/N, or about 0.70 in/N, or about 0.75 in/N.

In certain embodiments, the spring used in accordance with the devices described herein moves a total of 1 inch to go from a green indicator (i.e., a force of about 3 N) to a black indicator (i.e., a force of about 9 N). In other embodiments, the spring used in accordance with the devices described herein moves about 0.25 inches, or about 0.50 inches, or about 0.75 inches, or about 1.00 inches, or about 1.25 inches, or about 1.50 inches, or about 1.75 inches, or about 2.00 inches, or about 2.25 inches, or about 2.50 inches, or about 2.75 inches, or about 3.00 inches, or about 3.25 inches, or about 3.50 inches to go from a green indicator (i.e., a force of about 3 N) to a black indicator (i.e., a force of about 9 N).

In certain embodiments, the compression springs used in devices described herein have a Hooke's constant of 2.05 lb/in or 359 N/m and gives a throw of 0.11 inches per Newton, with 0.99 inches of displacement at 9 N.

In some embodiments, devices described herein further comprise a locking mechanism. In some embodiments, the locking mechanism is engaged by twisting the handle (300) counterclockwise. In some embodiments, the locking mechanism is disengaged by twisting the handle (300) clockwise. In other embodiments, the locking mechanism is engaged by twisting the handle (300) clockwise and disengaged by twisting the handle (300) counterclockwise.

In some embodiments, the locking mechanism is unidirectional. In other embodiments, the locking mechanism is bidirectional.

In some embodiments, the locking mechanism is a mechanical locking mechanism. In other embodiments, the locking mechanism is a friction-based locking mechanism. In further embodiments, the locking mechanism is a detent locking mechanism. In some embodiments, the locking mechanism is a ratchet mechanism. In some embodiments, the mechanical locking mechanism is a ratchet mechanism. In other embodiments, the locking mechanism is a spring loaded latch mechanism. In some embodiments, the locking mechanism is a magnetic locking mechanism. In other embodiments, the locking mechanism is a magnetic assisted locking mechanism.

As used herein, a “mechanical locking mechanism” refers to a mechanism which uses some kind of obstruction of a part by another part. As used herein, a “friction-based locking mechanism” refers to a mechanism which uses friction in order to prevent motion between two parts. As used here, a “ratchet mechanism” refers to a mechanism that consists of a bar or wheel having inclined teeth into which a pawl drops so that motion can be imparted to the wheel or bar, governed, or prevented, and allows unidirectional movement (linear or rotary). In some embodiments, a ratchet locking mechanism is a mechanical locking mechanism. As used herein, a “detent locking mechanism” refers to a mechanism that uses mechanical and/or magnetic means to resist or arrest the rotation of a wheel, axle, or spindle.

In some embodiments, the locking mechanism is a pawl and ratchet mechanism. In some embodiments, the pawl and ratchet mechanism is engaged by twisting the handle (300) counterclockwise. In some embodiments, the pawl and ratchet mechanism is disengaged by twisting the handle (300) clockwise. In other embodiments, the pawl and ratchet mechanism is engaged by twisting the handle (300) clockwise and is disengaged by twisting the handle (300) counterclockwise.

In some embodiments, when the locking mechanism is engaged and the handle (300) of the plunger (200) is pushed towards the first end (110) of the cylindrical outer casing (100), the pawl of the locking mechanism interacts with a ratchet at predetermined intervals. In some embodiments, the predetermined intervals correspond to applied forces of 3 N, 6 N, or 8 N. In other embodiments, when the locking mechanism is engaged, and the handle (300) of the plunger (200) is pushed towards the first end (110) of the cylindrical outer casing (100), the pawl of the locking mechanism interacts with a ratchet at a single predetermined interval. In some embodiments, the predetermined intervals correspond to applied forces of 6 N.

In certain embodiments, mechanical devices described herein comprise detents or triggered sub-mechanisms to provide a mechanical auditory and/or tactile alert (mechanical click or tone) upon an applied force. In some embodiments, these systems may be auto-resetting or may require a reset to be applied after a specific indication value is reached. This mechanism may or may not include lockouts of mechanism travel at a specified applied force value requiring a reset to resume device usage. In other embodiments, no user-capable reset is possible, providing a tell-tale indication of over force. In one embodiment, a resettable indicator provides a locking click at a maximum applied force. In further embodiments, a variation of devices described herein use light detents to provide intermediate force indication values. In some embodiments, the devices described herein may be reset by turning the handle (300) clockwise. In other embodiments, the devices described herein may be reset by turning the handle (300) counterclockwise.

Without wishing to limit the present invention to any theory or mechanism it is believed that detent force is within a pre-specified error value at a fraction of a Newton such that the impact on measurement is negligible.

In some embodiments, the devices described herein may be used without engaging the locking mechanism. Without wishing to limit the present invention to any theory or mechanism, it is believed that using the devices described herein without engaging the locking mechanism allows for bidirectional movement of the device along a guidewire and allows for the indicators (e.g., visual indicators) to reflect real-time forces.

In some embodiments, the devices described herein may be used in two modes. A mode (i.e., mode 1) in which the locking mechanism is engaged and locks (i.e., ratchets forward) at predetermined intervals and another mode (i.e., mode 2) which allows for free bidirectional motion of the device. In some embodiments, a user may freely toggle between the two modes by rotating the handle (300) relative to the barrel (e.g., clockwise or counterclockwise). In some embodiments, mode 2 is used to reset the device.

In some embodiments, a variation of devices described herein comprise a rotary type click is used whereby raised points to actuate ramps on a cam causing rotation to generate clicks to provide alerts for predetermined force levels. This mechanism is similar to the type used in a retractable ballpoint pen except with multiple rotational actuations per linear travel corresponding to different indication values. In other embodiments, a variation of devices described herein comprise tuned tines are aligned with detents on the device such that these tines are “plucked” when passed by a detent well, thereby producing tactile resistance as well as distinct auditory tones as they are traversed. In further embodiments, a variation of devices described herein comprise a coaxial alert mechanism driven with multiple spring-based mechanisms are triggered for each force value producing a loud indication sound. In some embodiments, a reset action is required to reset after each triggered actuation. This system may or may not lock out at individual steps passed or at the ultimate force indication step.

In further embodiments, an electromechanical variation of the devices described herein may trigger electrical driven alerts, including voice annunciation, tones, piezo indicators, vibration, or electrical-based triggering of mechanical type feedback mechanisms at predetermined measured forces.

In some embodiments, the compression of the spring (400) corresponds to an applied force. In other embodiments, the displacement of the spring (400) corresponds to an applied force. In further embodiments, the displacement of the spring (400) is mechanically coupled to an indicator. In some embodiments, the displacement of the spring (400) is mechanically coupled directly to an indicator. In other embodiments, the displacement of the spring (400) is coupled indirectly to an indicator through an assembly providing mechanical advantage or Vernier effect (i.e., the spring is mechanically or optically coupled to the indicator).

In some embodiments, the devices described herein further comprise indicators to alert the users visually, audibly, tactilely, or a combination thereof as the input force increases. In some embodiments, the indicators are visual indicators comprising a green visual indicator, a yellow visual indicator, a red visual indicator, a black visual indicator, or a combination thereof. In other embodiments, the indicators are visual indicators, comprising flashing green, yellow, and red lights. In further embodiments, the indicators are visual indicators comprising distinct and/or variable hues (e.g., multicolor lights) in which the position of the visual indicator matters rather than the color sequence of the visual indicators. Various colors may be used as visual indicators in accordance with the devices described herein. In some embodiments, the indicators are audible indicators, comprising increasing and/or disharmonious sounds as force increases.

In other embodiments, the devices described herein provide visual indicators, tactile indicators, audio indicators, or a combination thereof at three predefined force thresholds (e.g., 3 N, 6 N, 8 N) or at just one threshold (e.g., 6 N). In some embodiments, the devices described herein would further provide a color-coded, as well as auditory and tactile mechanical “click” when the first force threshold is reached, and another series of colors, sounds, and clicks as the second and third force is reached. In alternative embodiments, the devices described herein may only provide an indication when 6 N is applied to the UAS.

In some embodiments, the outer cylindrical casing (100) further comprises an indicator window. In some embodiments, the indicator window displays visual indicators corresponding to a force being applied. In some embodiments, the visual indicators comprise color indicators. In some embodiments, the indicator window displays visual indicators of green, yellow, red, black or a combination thereof. In some embodiments, a green visual indicator in the indicator window indicates 3 N of force being applied. In some embodiments, a yellow visual indicator in the indicator window indicates 6 N of force being applied. In some embodiments, a red visual indicator in the indicator window indicates 9 N of force being applied. In some embodiments, a black visual indicator corresponds to over 9 N of force being applied.

Indication of applied force is achieved via a window or indicator that provides alignment of a static indicator with the applied force. In one embodiment, this is achieved by a transparent window with a reticle type indicator aligning with an applied value marked on a moving indicator. Colors (green up to 3 N/yellow up to 6 N/red at 8 N or 9 N) can be used on the moving indicators behind the indicated force markings to provide awareness of the force being applied. In a related embodiment, mechanical advantage or an interference/Vernier type display can improve readability and visual precision measurement or clarity. In another embodiment (electromechanical type) a series of electrical contacts or linear potentiometer (or other type of displacement sensor including but not limited to hall-gate/magnetic, optical transmission or reflection, optical reflection) can be used to provide an electrical driven display that may include visual light indicators, a meter indicator, or similar type visual type for force applied.

In some embodiments, visual and tactile alerts (i.e., indicators) may be assisted by electronic means with optical, magnetic, resistive, switch-based measurement of spring displacement triggering electronic visual and/or tactile alerts.

In some embodiments, the force detecting, and force output is done continuously.

Without wishing to limit the present invention to any theories or mechanisms, it is believed that by alerting a surgeon to 6 N of force, allows for safe passage of the UAS (even up to 16 Fr). An 8-9 N indicator alerts a surgeon to a hard stop, because a force at that level will result in injury in upwards of one-fifth of cases that require a ureteral stent to be left in place for 2-6 weeks. The proposed device would enable the surgeon to avoid any injury to the ureter by adhering to a 6 N threshold. Further for UAS placed at the 3 N force threshold, surgeons may be able to safely eliminate ureteral stent placement after ureteroscopy; this alone would decrease the attendant morbidity and cost of ureteroscopic stone removals.

In some embodiments, the devices described herein further comprises a syringe barrel, wherein the cylindrical outer casing (100) is disposed inside and integrated with the syringe barrel.

In some embodiments, the load sensing devices described herein are not attached to the UAS but instead move freely along the guidewire.

The present invention features a device which may assist and provide a surgeon real-time feedback while deploying a ureteral access sheath (UAS) in the operating room, in order to preclude ureteral injury during positioning of the UAS. In some embodiments, devices described herein can be attached to a sidearm of a flexible ureteroscope to measure force applied as a ureteroscope is passed up the bare ureter (i.e., devoid of preplacement of an access sheath). In some embodiments, the devices described herein can either be built into the ureteral access sheath or passed over the guidewire until it abuts the end of the UAS. In one iteration, the device would have three indicators, at 3, 6, and 8 Newtons, or in a simpler iteration, it would have only one indicator, at 6 N. The former three levels of force would allow the user to know that if the UAS deploys at the first force threshold (i.e., indicator level 1 or 3 N), then a ureteral stent would not need to be left in place in an uncomplicated ureteroscopy. The second indicator (i.e., indicator level 2 or 6 N), would indicate to the user that a threshold force has been reached and the UAS should be downsized. The third indicator (i.e., indicator level 3 or 8 N), would inform the urologist that no additional force should be applied to the UAS as at this level, injury to the ureter is highly likely and thus the UAS should be downsized. This information would enable the urologist to pass the largest possible UAS yet preclude ureteral injury. The force indicators would be designed to provide visual, tactile and/or audio cues to provide alerts and operational feedback.

In some embodiments, a user is capable of continuously measuring input force in real time during the deployment of the surgical device in a patient.

In one embodiment, the present disclosure provides a load sensing device, comprising: a load cell adapted to receive a force, an input/output interface, and an adjustable attachment mechanism adapted to reversibly attach a surgical device, wherein the load cell detects the amount of applied force during the deployment of the surgical device in a patient and outputs a force value representative thereof via the input/output interface. In one embodiment, the force monitoring and force output is done continuously. In one embodiment, the device further comprises software adapted to continuously collect and store the force value. In one embodiment, the data collection and storage is done wirelessly. In one embodiment, the data collection and storage is done by Bluetooth™ technology. In one embodiment, the adjustable attachment mechanism is a screw port. In one embodiment, the adjustable attachment mechanism is a mechanical screw port. In one embodiment, the surgical device is a device used in an endoscopic, laparoscopic, robotic, and/or minimally invasive surgical procedure. In one embodiment, the surgical device is a ureteral access sheath. In one embodiment, the input/output interface is disposable. In one embodiment, a user is capable of measuring input force during the deployment of the surgical device in a patient. In one embodiment, the device is a handheld device. In one embodiment, the device further comprises indicators to alert the user as the input force increases. In one embodiment, the indicator is a visual indicator, comprising flashing green, yellow and red lights. In one embodiment, the indicator is an audio indicator, comprising increasing and/or disharmonious sound as the force increases.

In one embodiment, provided herein is a method of making a load sensing device, comprising: (a) providing a load cell, an input/output interface, and an adjustable attachment mechanism adapted to reversibly attach a surgical device; (b) connecting the load cell to the adjustable attachment mechanism such that the load cell receives and monitors the amount of applied force during the deployment of the surgical device in a patient; and (c) connecting the load cell to the input/output interface such that the input/output interface outputs a force value representative of the amount of applied force. In one embodiment, the force monitoring and force output is done continuously. In one embodiment, the device further comprises providing software adapted to continuously collect and store the force value. In one embodiment, the data collection and storage is done wirelessly. In one embodiment, the data collection and storage is done by Bluetooth™ technology. In one embodiment, the adjustable attachment mechanism is a screw port. In one embodiment, the adjustable attachment mechanism is a mechanical screw port. In one embodiment, the surgical device is a device used in an endoscopic, laparoscopic, robotic, and/or minimally invasive surgical procedure. In one embodiment, the surgical device is a ureteral access sheath. In one embodiment, the input/output interface is disposable. In one embodiment, the device is a handheld device. In one embodiment, the method further comprises indicators to alert the user as the input force increases. In one embodiment, the indicator is a visual indicator, comprising flashing green, yellow and red lights. In one embodiment, the indicator is an audio indicator, comprising increasing and/or disharmonious sound as the force increases. In one embodiment, sterile procedures are used throughout the manufacturing process.

In one embodiment, provided herein is a method of using a load sensing device during a surgical procedure of a patient comprising: (a) providing a load sensing device comprising a load cell adapted to receive a force; an input/output interface; and an adjustable attachment mechanism adapted to reversibly attach a surgical device; (b) providing a medical device to be inserted in the patient; (c) attaching the load sensing device to the medical device; and (d) inserting the medical device in the patient, wherein the load sensing device monitors and outputs the force value during the medical device insertion. In one embodiment, sterile procedures are used during the use process. In one embodiment, the medical device is a device used in an endoscopic, laparoscopic, robotic, and/or minimally invasive surgical procedure. In one embodiment, the medical device is a ureteral access sheath. In one embodiment, the load sensing device is a handheld device. In one embodiment, the load sensing device further comprises indicators to alert the user as the input force increases. In one embodiment, the indicator is a visual indicator, comprising flashing green, yellow and red lights. In one embodiment, the indicator is an audio indicator, comprising increasing and/or disharmonious sound as the force increases.

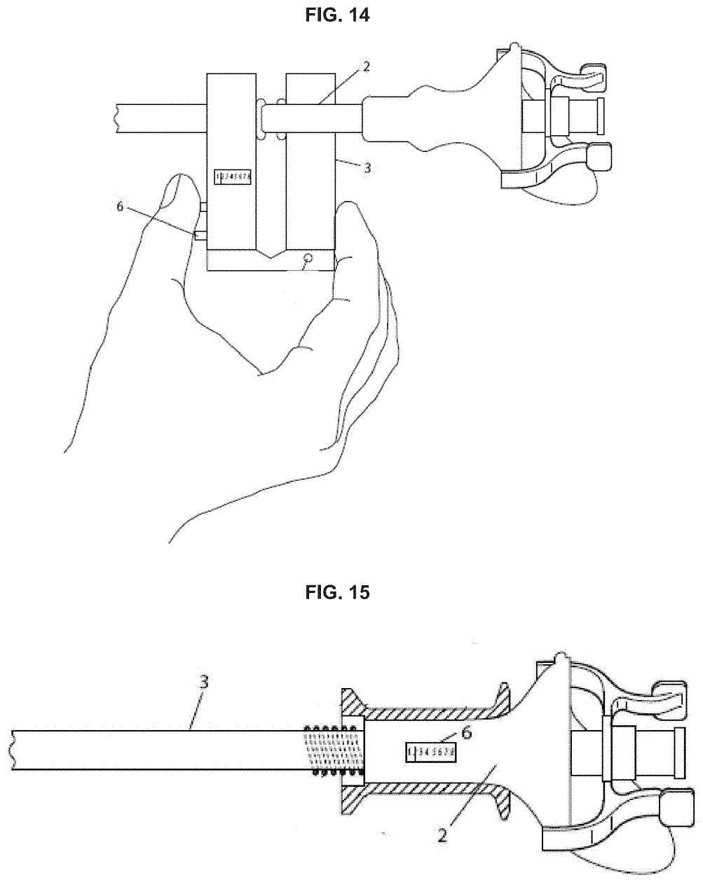

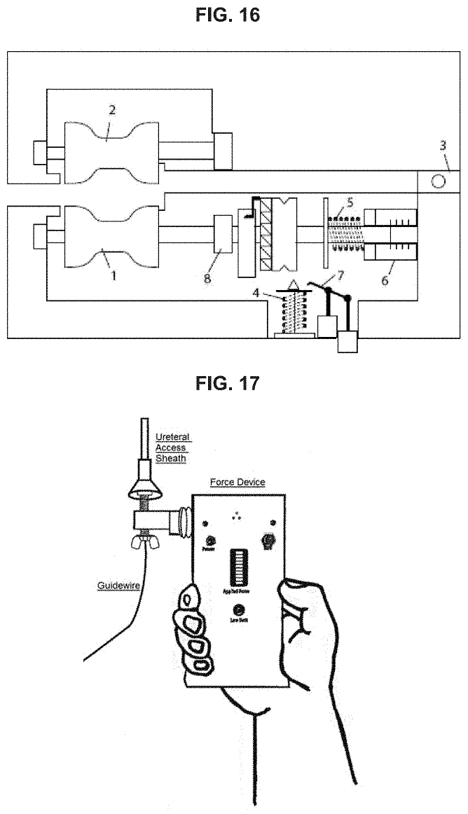

As described herein, in one embodiment, the device may be used for assisting and training surgeons by measuring force during ureteral access sheath (UAS) deployment in urological surgeries, as well as applicable to catheter and sheath insertion for urological, laparoscopic, robotic, and vascular procedures or for passage of a flexible ureteroscope into the bare ureter (i.e., devoid of an access sheath). Looking at FIG. 13, FIG. 14, FIG. 15, and FIG. 16 herein specifically, for example, one can see illustrated the device as either intrinsic with the access sheath or extrinsic as a device that can be slipped over any access sheath and used to measure force or slipped over a guidewire as a ureteroscope is advanced up the ureter over the guidewire or attached to the working port of the ureteroscope as it is passed up the bare ureter in a “NO Touch” technique. In accordance with various embodiments herein, for example, in one embodiment, the present invention provides a device where based on the information obtained from using a load cell, one could incorporate that information into a simple disposable device, for example, that was either intrinsic to the medical device, or UAS, or extrinsic to it, such as slipped over it, such that the operator would not be able to exert more than 8 N of force, for example, when passing the UAS or other devices or endoscopes into the human ureter.

In some embodiments, the devices described herein are mechanical devices. In other embodiments, the devices described herein are electromechanical.

In some embodiments, the present invention features a load sensing device comprises a load cell to receive a force, an input/output interface, and an adjustable attachment mechanism adapted to reversibly attach to a surgical device which is passed over a guidewire. In some embodiments, the load cell detects an amount of force applied to the butt end of a catheter during deployment of the surgical device over the guidewire in a patient and outputs a force value representative thereof through the input/output interface. In some embodiments, the surgical device is a ureteral access sheath (UAS). In some embodiments, the load sensing device further comprises indicators to alert the users in a visual and audible fashion as the input force increases. IN some embodiments, the load sensing device is not attached to the UAS but instead moves freely along the guidewire.

In some embodiments, the force detecting, and force output is done continuously. In some embodiments, the load sensing device described herein further comprises an external computer with software adapted to continuously collect and store the force value. In some embodiments, the force value collection and storage is done wirelessly. In some embodiments, the data collection and storage is done by ultra-high frequency radio waves limited in the 2.4 GHz band for wireless data transmission technology.

In some embodiments, the adjustable attachment mechanism is a screw port. In other embodiments, the adjustable attachment mechanism is a mechanical screw port. In further embodiments, the adjustable attachment mechanism is a magnetic screw port.

Without wishing to limit the present invention to any theories or mechanisms, it is believed that devices described herein may disengage from a surgical device (i.e., an access sheath (e.g., a ureteral access sheath (UAS)) or an endoscope) when the force gets too high. In some embodiments, the device is disengaged (i.e., releases) from the surgical device via a magnetic-based mechanism. In some embodiments, sensors are placed on the device at the interface for an access sheath (e.g., a ureteral access sheath (UAS)) or an endoscope.

In some embodiments, the sensor is a piezoresistive sensor (e.g., a piezoresistive strain gauge). As used herein, a “piezoresistive strain gauge” is a pressure sensor and uses the change in electrical resistance of a material when stretched to measure the pressure. In some embodiments, one piezoresistive sensor is used on the device at the interface for an access sheath (e.g., a ureteral access sheath (UAS)) or an endoscope. In other embodiments, two piezoresistive sensors are used on the device at the interface for an access sheath (e.g., a ureteral access sheath (UAS)) or an endoscope. In further embodiments, three piezoresistive sensors are used on the device at the interface for an access sheath (e.g., a ureteral access sheath (UAS)) or an endoscope. In some embodiments, four piezoresistive sensors, or five piezoresistive sensors, or six piezoresistive sensors, or seven piezoresistive sensors, or eight piezoresistive sensors, or nine piezoresistive sensors, or ten piezoresistive sensors are used on the device at the interface for an access sheath (e.g., a ureteral access sheath (UAS)) or an endoscope.

In some embodiments, the load sensing device is a handheld device. In some embodiments, the indicators are visual indicators, comprising flashing green, yellow, and red lights. In some embodiments, the indicators are audio indicators, comprising increasing and/or disharmonious sounds as force increases.

The present invention may further feature a load sensing device comprising a load cell adapted to receive a force, an input/output interface, and an adjustable attachment mechanism adapted to reversibly attach a surgical device which is passed over a guidewire. In some embodiments, the load cell detects an amount of force applied to the butt end of a catheter during deployment of the surgical device over the guidewire in a patient and outputs a force value representative thereof through the input/output interface. In some embodiments, the surgical device is a ureteral access sheath (UAS). In some embodiments, the load sensing device further comprises indicators to alert the user in a visual and/or audible fashion as the input forces increase. In some embodiments, the load sensing device is not attached to the UAS but instead moves freely along the guidewire. In some embodiments, the force detecting, and force output is done continuously. In some embodiments, the load sensing device further comprises an external computer with software adapted to continuously collect and store the force value. In some embodiments, the force value collection and storage is done wirelessly. In some embodiments, the input/output interface is used in a disposable arrangement. In some embodiments, the indicators are visual indicators, comprising flashing green, yellow, and red lights. In other embodiments, the indicators are an audio indicator, comprising increasing and/or disharmonious sound as the force increases.

The present invention may also feature a method of using a load sensing device during a surgical procedure of a patient. In some embodiments, the method comprises providing a load sensing device comprising a load cell adapted to receive a force, an input/output interface, and an adjustable attachment mechanism adapted to reversibly attach a surgical device that passes over a guidewire. In some embodiments, the method comprises providing the surgical device to be inserted in the patient. In some embodiments, the method comprises attaching the load sensing device to the surgical device. In some embodiments, the method comprises inserting the surgical device in the patient. In some embodiments, the load sensing device monitors and outputs force value applied to the butt end of a catheter during the surgical device insertion over the guidewire. In some embodiments, the surgical device is a ureteral access sheath (UAS). In some embodiments, the load sensing device further comprises indicators to alert the user as the input force increases. In some embodiments, the load sensing device is not attached to the UAS but instead moves freely along the guidewire.

### EXAMPLES

The following is a non-limiting example of the present invention. It is to be understood that said example is not intended to limit the present invention in any way. Equivalents or substitutes are within the scope of the present invention.

### Examples 1: Background and Results Relating to Pressure Monitoring During UAS Insertion

In one embodiment, the present disclosure provides a force measurement device for catheter and sheath insertion procedures. In one embodiment, the device can be directed towards urological sheath insertion force measurement.

The ureteral access sheath (UAS) was first introduced in the 1970's in order to provide an accessible conduit to the kidney for the rapid and safe repeated introduction and removal of an ureteroscope. Today, UAS is used commonly during flexible URS for stone management and to diagnose and treat other upper urinary tract diseases, such as strictures or upper urinary tract tumors. However, UAS is not universally embraced due to fear of ureteral injury, which may result from excessive force being applied during UAS placement. Currently, there are no devices that measure forces during UAS deployment; essentially, the UAS is deployed solely based on the surgeon's experience and sense of touch which leads to ureteral injuries in upwards of 20% of cases. In accordance with various embodiments herein, the device disclosed herein can be used for training and quality control/standardization purposes.

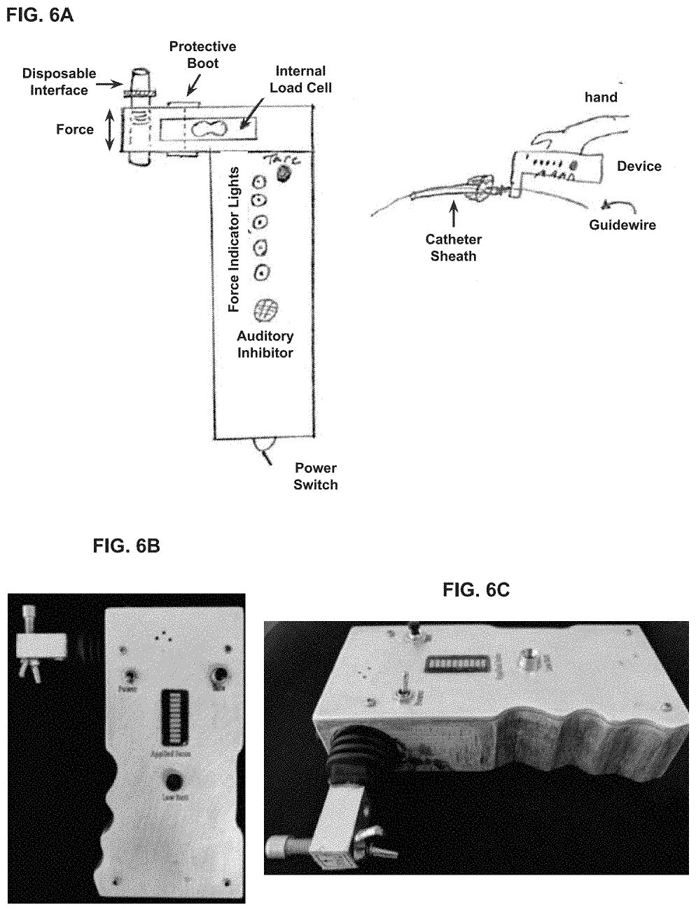

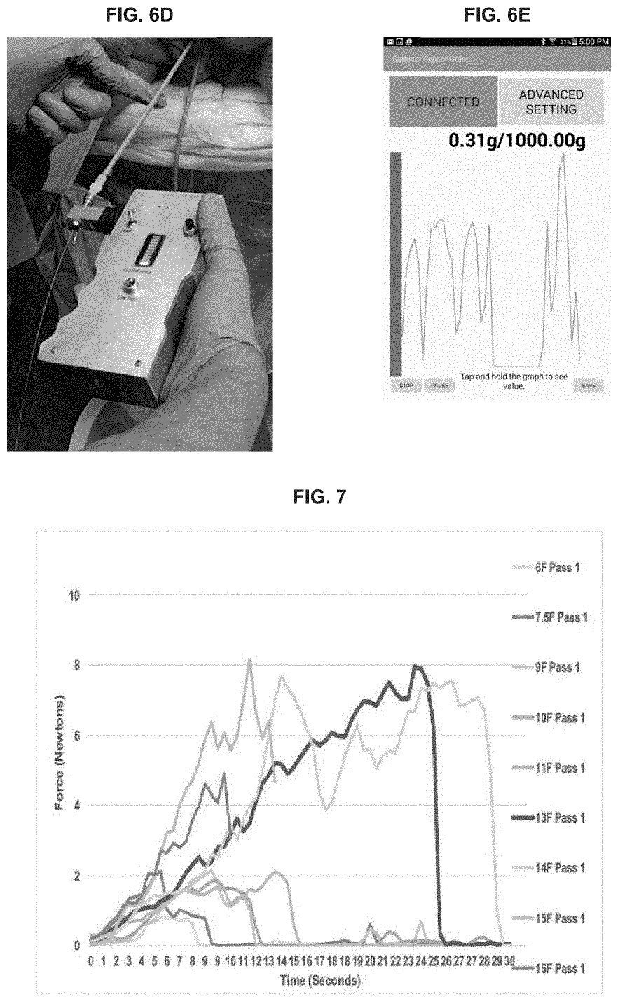

Currently there is no direct handheld system known to the inventors available in the market or discussed in the literature to help with manual catheter or insertion sheath deployment. As such, the inventors designed and developed a novel ureteral access sheath force sense device to be used in the operating room during any procedure requiring UAS insertion. FIG. 1 and FIGS. 6A, 6B, and 6C provide various embodiments of the devices described herein. The device will be connected to the UAS using sterile technique; for this a mechanical interface port (screw-port for a Luer-lock connector in the current design) housed in a sterile clear plastic bag is secured to the UAS. The device uses a load cell (based on multiple strain gauges) to measure force and would provide continuous measurements during deployment of the UAS. The device reports ad-hoc to the surgeon via visual and audio indicators of applied force. The handheld device component of the system links to an Android™ based tablet with a custom display and control application that interfaces with the device using Bluetooth™ technology that is connected to an external computer (running Android™ operating system) that will display the force in real time.

In one embodiment, as illustrated in FIGS. 6A, 6B, and 6C herein, the device is in a form factor to allow measurement of catheter sheath insertions via a side-mounted load cell. In another embodiment, the device is shaped to provide easy and ergonomic use in surgery. In one embodiment, the device is all metal and hermetically sealed providing water resistance for sanitization procedures and easy gas sterilization. In one embodiment, the device interfaces via Bluetooth™ technology to a tablet-based application to provide a GUI with controls. In one embodiment, the Bluetooth™ antenna is internally mounted near the rubber gasket to provide sufficient range while contained within an all-metal case construction.

In one embodiment, clinical studies were performed that for the first time define a safe range of force during ureteral access sheath deployment and define a range of excessive force that causes ureteral damage. Data from these experiments would further justify device usage but also should help to prevent injuries during surgical sheath insertion. In one embodiment, a sheath is developed with a purposely built-in failure mode such that use of excessive force results in buckling of the sheath distally and thus failure of the proximal end of the sheath to advance thereby shielding the ureter from exposure to excessive force. In one embodiment, the device is also contemplated to be used in procedures such as laparoscopy, robotics, vascular and interventional radiology procedures.

### Example 2: Background and Results Relating to Pressure Sensing Device

Ureteral injuries have been noted to occur during ureteral access sheath (UAS) deployment. The force exerted during deployment has yet to be measured; likewise, the amount of force that results in ureteral injury has not yet been quantitated. The inventors have developed a novel force-sensing device and tested its feasibility to identify, for the first time, the threshold force that reliably induces a ureteral injury in a porcine model: their studies have now been corroborated in over 200 clinical cases.

With Institutional Animal Care and Use Committee (IACUC) approval, ureteral instrumentation (ureteral dilator and UAS) deployment force was measured using the Ureteral Access Sheath Force Sensor (UAS-FS) device disclosed herein. The force exerted was measured continuously from when the tip of the access sheath was introduced at the urethral meatus to when the distal end of the dilator was hubbed at the urethral meatus; insertion of the sheath was monitored fluoroscopically. Each catheter/sheath (6-16 F) was passed twice. Ureteroscopic evaluation was performed with each catheter/sheath after the 9.5 F obturator was passed in order to look for potential ureteral injuries. Injuries were graded using the Post-Ureteroscopic Lesion Scale (PULS, from 0—no injury to 5 severe ureteral disruption).

No injuries were detected when the deployment force remained under 4 Newtons (N) which was the case when the UAS was ≤13 F. Increasing UAS size above 13 F resulted in larger force measures during UAS passage and greater peak forces. First ureteral injury was seen with a peak force of 8 N and 10 N in the right and left ureters, respectively in the initial porcine study.

The UAS-FS can measure force applied to a catheter or sheath in a reproducible fashion. Peak force <4 N was found to be safe during UAS deployment in a porcine model. One-time peak force ≥8 N resulted in ureteric injury. A clinical study proposal has been approved by the UC Irvine IRB and has now been completed showing that similar forces apply in the human ureter. At <6 N there were no clinical Grade 3 injuries to the ureter; PULS 3 injuries were noted when >8 N was applied in an attempt to pass the UAS.

### Example 3 Generally

Flexible ureteroscopy (URS) has become a method of definitive management for small ureteral and upper urinary tract calculi. The ureteral access sheath (UAS) was developed to facilitate the repeated passage of the flexible ureteroscope. Use of the UAS is associated with decreased intrarenal pressures, more efficient rate of stone removal, reduced operative time, and a reduction in ureteroscope repair costs.

Although the UAS has undergone design modifications to improve usability (e.g., size variety, reductions in buckling and kinking, and a hydrophilic coating) its use is not yet widespread. Stress forces exerted upon the ureteral wall during UAS deployment have raised concerns. A prospective investigation by Traxer et al estimated the incidence of UAS-induced ureteral injury with passage of a 14 F UAS to be as high as 48%, with serious injuries (i.e. ureteral perforation/splitting—PULS 3) noted in 13% of clinical cases; more recently Monga and associates recorded a 23% incidence of PULS 3 injuries with passage of a 14 F UAS (Loftus, CS, Monga, M, et al. JE: 34: 932, 2020)

An established threshold of application force and a method of real-time force measurement during UAS deployment could markedly improve the safety of using UAS during URS.

In an effort to characterize the forces exerted upon the ureteral wall during UAS deployment, the inventors have developed a novel load-sensing device, the UCI Ureteral Access Sheath Force Sensor (UAS-FS). In one embodiment, the UAS-FS device disclosed herein would allow a user to measure the applied forces, identify a safe range of forces, and define a threshold force beyond which UAS-induced damage would be most likely to occur.

### Example 4: Methods

Ureteral Access Sheath Force Measuring Device Development: The Ureteral Access Sheath Force Sensor (FIGS. 6A, 6B, and 6C) device was designed for sterile use in the operating room during endoscopic procedures requiring placement of a UAS. An internal load cell is used as a strain gauge to measure input force during UAS deployment. The device is equipped with a disposable interface, in which a mechanical screw-port (i.e., Luer-Lock™ mechanism) can be secured to the UAS using sterile technique. Bluetooth™ technology and device specific software allow for continuous wireless data collection (i.e., pound-force (lbf) or gram-force (gf)) via an external wireless device (such as Android™ device). All UAS-FS measurements were converted to Newtons (N) (1 N=0.225 lbf=101.97 gf). In addition, the device provides both real-time visual (via force indication lights from green (low force) to yellow to red (excessive force)) and audio (increasing sound) graded feedback to alert the user as the input force increases. The device can be sterilized using a STERRAD system (Ethicon US, LLC).

Animal Preparation: Following approval by the Institutional Animal Care and Use Committee (IACUC), a 35-kilogram female Yorkshire pig was placed under general anesthesia. Vital signs, temperature, and muscle tone were continuously monitored throughout the procedure as per the Guide for the Care and Use of Laboratory Animals.

Preliminary Ureteral Access: With the animal supine, the pelvis was prepared with povidone-iodine solution and standard drapes were applied. A flexible cystoscope (11272 VP, Karl Storz Inc., Germany) was introduced into the urethra and a 0.035 in. guidewire (Amplatz Super Stiff Guidewire, Boston Scientific Corp., Natick, Mass.) was placed into the left ureteral orifice and fluoroscopically guided to the kidney. Once studies with varying sizes of dilators/UAS were done with the force sensor on the left ureter, the procedure was repeated on the right ureter.

Ureteral Dilator and Access Sheath Deployment Forme Measurement and Assessment of Ureteral Damage: A single individual (KK) performed all ureteral dilator and UAS deployments over the guidewire using the UAS-FS. Force measurement was initiated upon insertion of the UAS tip at the urethral meatus and stopped when the UAS was either hubbed at the meatus or the tip of the device had reached the renal pelvis; the entire procedure was done under fluoroscopic guidance. Measurements were recorded using several ureteral dilators (6 F, 7.5 F, 8 F, 9 F) and UAS obturators and their respective sheath (9.5/11 F, 35 cm; 10/12 F, 35 cm; 11/13 F, 36 cm; 12/14 F, 35 cm; 13/15 F, 36 cm; 14/16 F, 35 cm) (Cook Medical® Inc., Bloomington, Ind. & Boston Scientific Corp., Natick, Mass.). Forces were recorded twice (i.e., Pass 1 and Pass 2) for both the right and left ureter, respectively. After the passage of the 9.5 F obturator, flexible ureteroscopy (Flex-X2, Karl Storz Inc., Germany) was performed for each subsequent passage of larger dilators/obturators/sheaths to assess for ureteral injury. The appearance of a ureteral injury was recorded, and the location confirmed with fluoroscopy. Injuries were graded using the Post-Ureteroscopic Lesion Scale (PULS) on a scale of “0” no injury to “5” complete ureteral disruption. The porcine bladder was emptied between deployments using a 10 F Foley catheter.

### Example 5: Results and Analysis

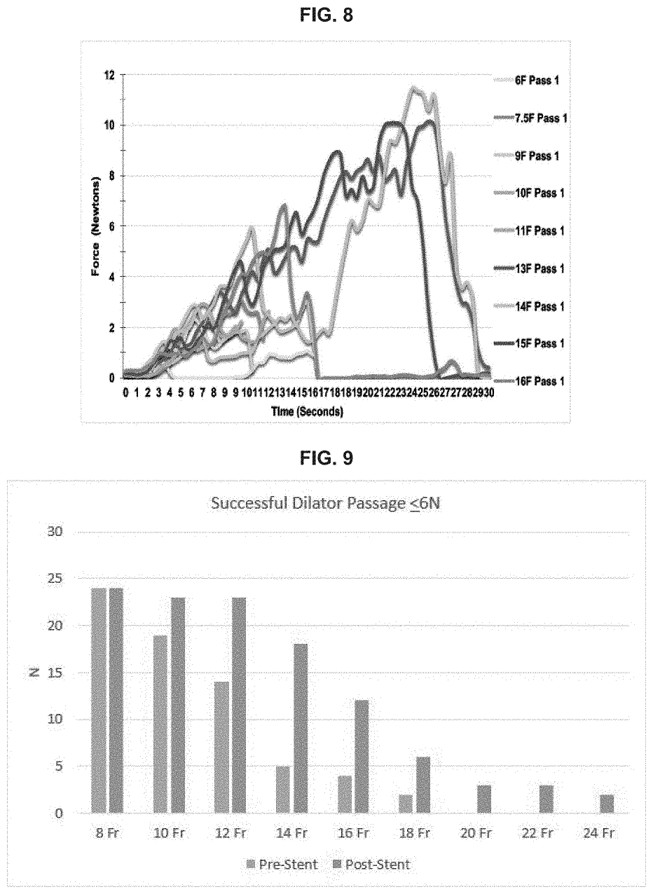

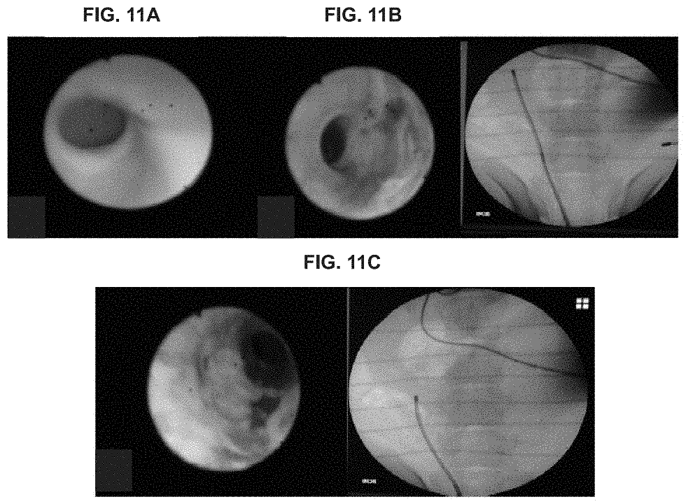

In one embodiment, force measurements were performed during ureteral dilator and access sheath deployment. The measured deployment force increased with larger diameter UAS in both the right and left ureter (FIG. 7 and FIG. 8, respectively). The mean application force for each insertion remained relatively steady until deployment of the 13 F UAS (FIG. 9). The mean application force of the second pass was often lower than the first. Both the mean and peak application force notably increased following placement of the 13 F UAS (FIG. 11). The greatest change in mean application force (>1 N) between the passes was seen with UAS a 13 F. Similarly, the peak application force was notably higher during deployment of UAS, 11.5 F.

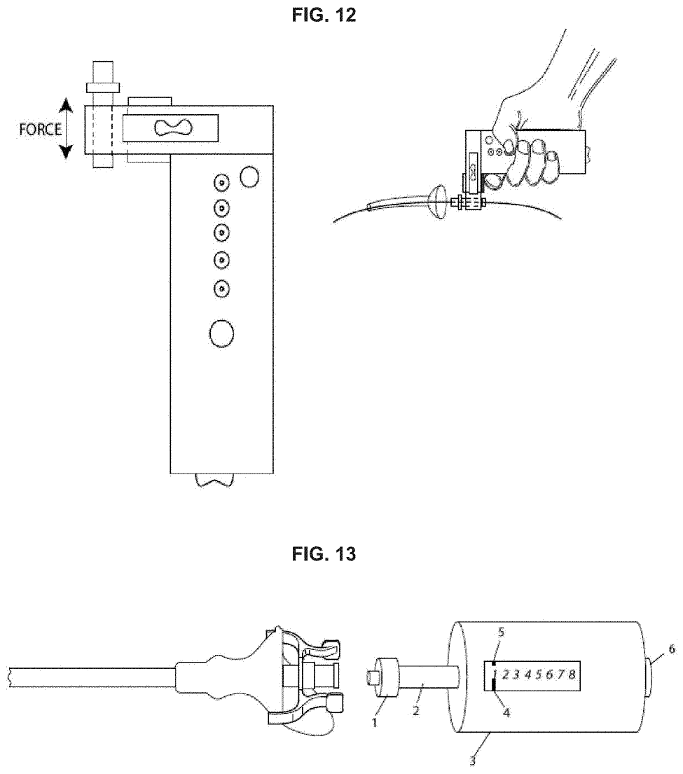

Ureteral injuries were noted during placement of the 13 F UAS, with a peak force of 8 N on the right ureter and 10 N on the left (FIG. 12 & FIG. 14). These injuries were both graded as a PULS 3. Subsequent PULS 4 injuries to the right ureter occurred following placement of the 14 F UAS. Although a peak force of 4.8 N was recorded, further injury (PULS 4) to the right ureter was visualized during deployment of the 16 F UAS, likely due to the fact that the urothelium had already been split and the integrity of the ureteral wall once breached, resulted in further spreading of the edges with minimal force applied. Images and corresponding fluoroscopic location of ureteral injuries are demonstrated in FIG. 16.

Generally, the ureteral access sheath has faced scrutiny since its introduction due to urothelial injury and potential concerns over subsequent ureteral stricture formation. Concerns were reported in the literature with the original UAS upon discovering a ureteral perforation in 8 of 43 (19%) cases in a prospective study (Newman et al, 1987). Numerous design changes occurred over the following decade (e.g., tapered proximal tip of the inner dilator, metal coiling of the outer sheath to preclude kinking, and a liquid-activated hydrophilic coating) to decrease the risk of ureteral injury. Nevertheless, in a prospective investigation of 359 patients undergoing UAS-facilitated URS, low-grade ureteral injury was reported in 86% of patients and high-grade injury in 13% of patients. (Traxer et al). The force required to induce such injuries has not been defined and thus one is left with only an anecdotal, empirical sense of how hard to push on the UAS in order to position it.

In this seminal study, the inventors aimed to define how much force could be applied to the ureteral wall before it would split. To this end, the inventors have developed a novel load-sensing device (UAS-FS) that could be used in the operating room to measure the applied force throughout the deployment of a catheter or UAS over a guidewire. In this study 8 N of force resulted in injury to the adult female porcine ureter. When forces remained under 4 N no injury was observed. As the diameter of the UAS increased so did the force required to deploy it; however, it was not until a 13 F sheath was passed that 8 N was reached and a ureteral injury was noted. Not surprisingly, once the ureter was “dilated” from the first pass of the 13 F sheath, during the second pass there was less force needed to deploy the same sized UAS. Of importance, once an injury had occurred, less force was required to induce further injury.

The concept of evaluating force placed on the ureter during rigid ureteroscopy (URS) was first introduced by Ulvik et al.; they created a novel coupling device to attach a standard force meter to an 8.5 F semirigid ureteroscope to measure forces exerted in the ureter during rigid URS. Mean retrograde insertion force ranged from 9.7±7.3 N at the distal ureter to 4.4±3.6 N at the proximal ureter. Although no complications were reported, the Ulvik study did not grade ureteral injury nor provide long-term patient follow-up. They also did not apply their device to the passage of a UAS or a flexible ureteroscope.

Similar studies utilizing porcine models have been performed to assess the force of UAS deployment. A feasibility study by Harper et al., compared the insertion force following placement of a novel radially dilating 9.5 F and a conventional 12/14 F UAS in a porcine model to the level of the UPJ. The researchers reported mean forces of 0.48 and 2.2 N, respectively, and maximum forces of 1.6 and 6.5 N, respectively. These results are similar to the current findings using the UAS-FS during 9.5 F and 12/14 F UAS deployment (mean application force of 0.67 and 3.63 N, respectively), however peak application force showed greater variation (1.4 to 2.9 N and 7.6 to 11.4 N, respectively). Furthermore, although the Harper study used a novel trauma score to assess ureteral damage (n.b.: not PULS) following UAS placement, the amount of force at the moment of injury was not provided. Of note, like Ulvik and colleagues, they used a standard force sensor that was not designed specifically for force measurement during passage of a catheter or sheath deployed over a guidewire.

Lidal et al. evaluated the effect of isoproterenol on the insertion force of placing a 13/15 F UAS in the porcine ureter, again using a standard force sensor with a coupling mechanism. During UAS placement, input force was measured, and deployment was stopped once subjective resistance was met by the operator. Subsequently, the pharmacological intervention (i.e., isoproterenol vs. saline) via a 10 F ureteral catheter was delivered. Input force for ureters treated with isoproterenol was significantly less than those irrigated with saline. Importantly, mean endpoint values 5.34 N (pre-irrigation) and 4.6 N (in 6 ureters with no subjective resistance) are within limits of mean (1.7-4.0) and peak (4.2-10.0) 15 F UAS forces measured in this instant study with the UAS-FS. Their study corroborated that the force applied, and resulting ureteral injury, varied with the operator thus highlighting the importance of being able to actively measure application force during UAS deployment in real time. Their study did not measure forces for different sized UAS or define a threshold for injury.

The study and results disclosed herein provide reliable continuous measurements in a clinical setting. In the most recent clinical study reported by the inventors, the force exerted to pass a UAS was determined in 210 ureters in 200 patients. In this study a PULS of </=2 occurred at 6 N and PULS of 0-1 was recorded at </=5 N. In two cases a PULS 3 injury was noted; in both cases this occurred when 8 N was exceeded. A standard force transducer was not used for comparison with our novel UAS-FS because conventional force transducers are not specifically designed to be used over a guidewire.

The novel UCI UAS-FS accurately and continuously measures forces during ureteral dilator or UAS deployment in a reproducible manner in an adult female pig. A peak force of less than 4 N was not associated with any ureteral injuries: however, a single, even brief application of 8 N force was sufficient to cause a ureteral injury. These results were corroborated in a clinical study involving 200 patients and 210 ureters (Tapiero S, Kaler K S, Jiang P, Lu S, Cottone C, Patel R M, Okhunov Z, Klopfer M J, Landman J, and Clayman R V: Determining the Safety Threshold for the Passage of a Ureteral Access Sheath in Clinical Practice Using a Purpose-Built Force Sensor. J Urol. 206(2): 364-372, 2021)

### Example 6 Impact of Force Applied During UAS Deployment on Ureteral Injury in a Porcine Model

Widespread use of the ureteral access sheath (UAS) during ureteroscopy has been slowed by concerns over possible ureteral injury during its passage. In this porcine study, using the novel device disclosed herein, the inventors evaluated the force threshold which would induce ureteral injury.

The inventors measured UAS deployment force using a novel Ureteral Access Sheath Force Sensor (UC Irvine Force Sensor) in a female Yorkshire pig. Under fluoroscopic control, force was continuously measured from the time the UAS contacted the urethral meatus until the tip of the UAS had reached the renal pelvis. Ureteral dilators (6-9 F), UAS and its obturators (9.5 F. 10 F, 11 F, 11.5 F, 12 F, 13 F, 14 F, 15 F, 16 F) sequentially passed twice into both ureters. Ureteroscopic evaluation was initiated after the 9.5 F UAS obturator was passed. No ureteral injury occurred at <4 Newtons (N). Increasing UAS size resulted in greater force over-time and larger peak forces (FIG. 14). First ureteral injury occurred at 8 N (right ureter) and 10 N (left ureter). FIG. 16 shows a normal right ureter before and after deployment of a 13 F UAS with a peak force of 8 N. Accordingly, the force sensor device disclosed herein can reliably and continuously measure force while deploying a UAS. Initial ureteral injury occurred at forces >8 N.

### Example 7 UAS Force Device Experiment Summary

Concerns regarding ureteral injury have limited the more widespread use of a ureteral access sheath (UAS) during ureteroscopy. The amount of force during ureteral access sheath deployment that results in clinical ureteral injury has not been defined. Two experiments were performed using the ureteral access sheath force sensor device (FIG. 12 and FIG. 14). The inventors performed a follow-up porcine study with more extensive testing of the novel UAS Force Sensor (UAS-FS). They also completed clinical testing of the sensor during routine ureteroscopy and percutaneous nephrolithotomy in stone patients.

Porcine Model Experiment: The UAS-FS was used to measure UAS deployment forces in six female Yorkshire pigs (average size 19.5 kg (16-22 kg). Under fluoroscopic guidance, ureteral dilators (6-9 F) along with a variety of UAS and corresponding obturators (9.5 F-16 F) were sequentially advanced into one of the ureters in each pig. In the other ureter, after 8/10 F dilation, the 12/14 F UAS was deployed without any sequential dilation. Force was measured continuously from the time the UAS entered the urethral meatus until the tip of the UAS reached the renal pelvis. The bladder was drained between deployments as needed. Ureteroscopic evaluation was performed after each dilation to assess for ureteral injury using the post ureteroscopic lesion scale (PULS) ranging from 0 (no injury) to 5 (complete ureteral disruption).

No PULS 3 ureteral injury (i.e., splitting of the urothelium with exposure of suburothelial fat) was observed at forces ≤5.5 Newtons (N). Increasing UAS diameter resulted in larger peak forces. Ureters subjected to sequential dilation did not incur a significant injury (i.e., PULS 3) until a peak force of 8.83 N was reached. The UASFS reliably measures force while deploying a UAS. Initial ureteral injury within these smaller pigs was avoided provided the peak force remained <5.5 N.

Clinical Experiment: In the initial porcine study, the inventors noted that a peak force of 8 Newtons during ureteral access sheath deployment resulted in splitting of the ureter. With this information, the inventors began clinical testing using UAS-FS (FIG. 17) during routine ureteroscopy and percutaneous nephrolithotomy in stone patients.

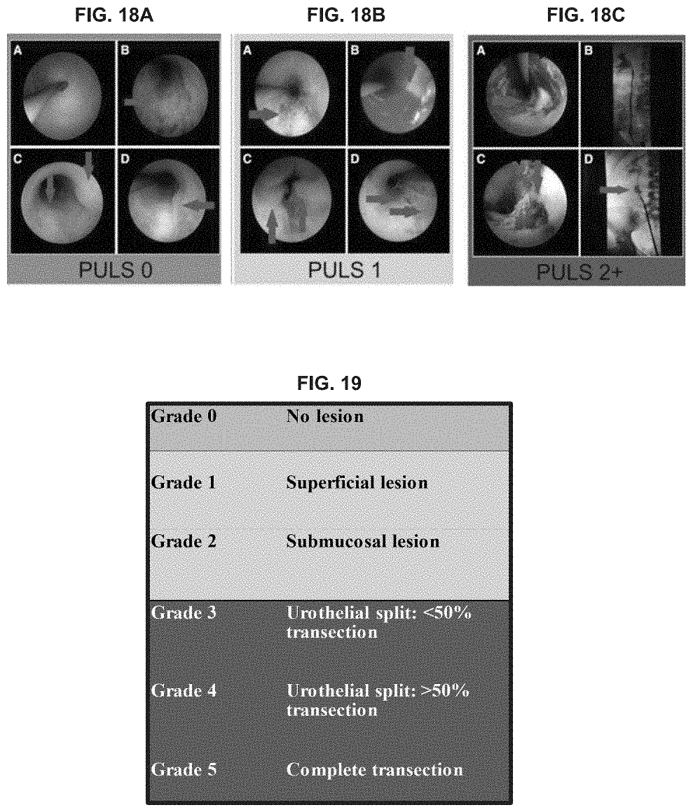

UAS deployment force was measured in 200 patients using UAS-FS under fluoroscopic control by 4 different surgeons. Tamsulosin was given for up to one week in two-thirds of these patients to induce a state of ureteral relaxation. Continuous UAS-FS measurements began from when the tip of the UAS was inserted into the urethra until the tip of the UAS reached the ureteropelvic junction. If the force approached/began to exceed 8 N (audible sound), passage was stopped, progress of the UAS was recorded fluoroscopically, and the UAS was withdrawn and a smaller UAS selected. The physicians were cautioned to not exceed 6 N. Ureteroscopic evaluation of the entire ureter was performed at the end of each case to assess for potential ureteral injuries using the post-ureteroscopic lesions scale (PULS) (Table 1) (FIGS. 18A, 18B, and 18C).

Among the 200 patients, there were 210 UAS deployments. The 16 French UAS could be passed at a mean peak pressure of 5.7 N in 61% of patients; in the remainder, a smaller UAS was deployed (14 F in 61 cases and 11.5 F in 22 cases) being careful to not exceed the recommended force of 6 N. Two PULS 3 injuries occurred; in both cases a force of 8.0 N and 8.4 N was recorded. Of note, in the study, 8 N was exceeded in 39 instances of which 2 resulted in a PULS 3 injury (5.1%). Furthermore, a </=5 N, the PULS score was 0-1. The UAS-FS was able to measure UAS insertion force in a reproducible fashion. Limiting the force exerted on the UAS to <8 N, precluded a PULS score of 3 in all patients.

### Example 8

TABLE 1
post ureteroscopic lesion scale (PULS; Grade 0-5)

| | | |
|---|---|---|
| Grade 0 | No lesion | Uncomplicated URS |
| Grade 1 | Superficial mucosal lesion | (no grading according to the |
| | and/or significant mucosal | Dindo-modified Clavien |
| | edema/hematoma | classification of surgical |
| | | complications) |
| Grade 2 | Submucosal lesion | |
| Grade 3 | Perforation with less than | Complicated URS (Grade 3a |
| | 50% partial transection | or b according to the |
| | | Dindo-modified Clavien |
| | | classification of surgical |
| | | complications) |
| Grade 4 | More than 50% partial | |
| | transection | |
| Grade 5 | Complete transection | |

The various methods and techniques described (vide supra) provide a number of ways to conduct the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of,” and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

### Embodiments

The following embodiments are intended to be illustrative only and not limiting in any way.

Embodiment 1: A handheld load sensing device, comprising: (a) a cylindrical outer casing (100), wherein the cylindrical outer casing (100) comprises a first casing end (110), a second casing end (120), an exterior lumen (131), and a guidewire lumen (132) disposed within the exterior lumen (131), wherein the first casing end (110) comprises a first opening (111) and the second casing end (120) comprises a second opening (122) for accessing the guidewire lumen (132); (b) a plunger (200) slidably coupled to the cylindrical outer casing (100), wherein the plunger (200) comprises a first plunger end (210) and a second plunger end (220) having a handle (300) disposed thereon, wherein the first plunger end (210) is disposed through the first opening (111) of the first casing end (110) and within the exterior lumen (131), wherein the first plunger end (210) comprises a plunger lumen (230), wherein the plunger lumen (230) and the guidewire lumen (132) of the cylindrical outer casing (100) are fluidly coupled; and (c) a spring (400) disposed within the exterior lumen (131) of the cylindrical outer casing (100), wherein a first spring end (410) of the spring rest against the second casing end (120) and a second spring end (420) rest against the first plunger end (210), wherein the guidewire lumen (132) is disposed through the spring (400) such that the spring (400) wraps around the guidewire lumen (132); wherein when the handle (300) of the plunger (200) is pushed towards the first casing end (110), the plunger (200) pushes against and compresses the spring (400), wherein displacement of the spring (400) corresponds to an applied force.

Embodiment 2: The device of embodiment 1, wherein the second casing end (120) of the cylindrical outer casing (100) further comprises an interface for a ureteral access sheath (UAS) or an endoscope.

Embodiment 3: The device of embodiment 2, wherein the endoscope is flexible.

Embodiment 4: The device of embodiment 2, wherein the endoscope is rigid.

Embodiment 5: The device of embodiment 2, wherein the interface comprises a Luer-lock style interface

Embodiment 6: The device of embodiment 2 or embodiment 5, wherein the interface is positioned distal to a point of insertion of the UAS or the endoscope.

Embodiment 7: The device of any one of embodiments 1-6, further comprising a locking mechanism.

Embodiment 8: The device of embodiment 7, wherein the locking mechanism is a pawl and ratchet mechanism.

Embodiment 9: The device of embodiment 8, wherein the pawl and ratchet mechanism is engaged by twisting the handle (300) counterclockwise.

Embodiment 10: The device of embodiment 8, wherein the pawl and ratchet mechanism is disengaged by twisting the handle (300) clockwise.

Embodiment 11: The device of any one of embodiments 1-10, wherein the outer cylindrical casing (100) further comprises an indicator window.

Embodiment 12: The device of embodiment 11, wherein the indicator window displays visual indicators corresponding to a force being applied.

Embodiment 13: The device of embodiment 12, wherein the visual indicators comprise color indicators.

Embodiment 14: The device of embodiment 12 or embodiment 13, wherein the visual indicators comprise a green visual indicator, a yellow visual indicator, a red visual indicator, a black visual indicator, or a combination thereof

Embodiment 15: The device of embodiment 12 or embodiment 13, wherein a green visual indicator corresponds to about 3 N of force being applied.

Embodiment 16: The device of embodiment 12 or embodiment 13, wherein a yellow visual indicator corresponds to about 6 N of force being applied.

Embodiment 17: The device of embodiment 12 or embodiment 13, wherein a red visual indicator corresponds to about 9 N of force being applied.

Embodiment 18: The device of embodiment 12 or embodiment 13, wherein a black visual indicator corresponds to over 9 N of force being applied.

Embodiment 19: The device of any one of embodiments 1-18, wherein the device is made of plastic.

Embodiment 20: The device of any one of embodiments 1-18, wherein the device is made of metal.

Embodiment 21: The device of any one of embodiments 1-20, further comprising a syringe barrel, wherein the cylindrical outer casing (100) is disposed inside and integrated with the syringe barrel.

Embodiment 22: A handheld load sensing device, comprising (a) a force sensor to receive a force; (b) an input/output interface, and (c) an adjustable attachment mechanism adapted to reversibly attach to a surgical device which is passed over a guidewire; wherein the force sensor detects the amount of force applied to the butt end of a catheter during deployment of the surgical device over the guidewire in a patient and outputs a force value representative thereof through the input/output interface.

Embodiment 23: The load sensing device of embodiment 22, wherein the force sensor is mechanical.

Embodiment 24: The load sensing device of embodiment 23, wherein the force sensor is a spring.

Embodiment 25: The load sensing device of embodiment 22, wherein the force sensor is electromechanical.

Embodiment 26: The load sensing device of embodiment 25, wherein the force sensor is a load cell.

Embodiment 27: The load sensing device of embodiment 22, wherein the surgical device is a ureteral access sheath (UAS) or an endoscope.

Embodiment 28: The load sensing device of embodiment 27, wherein the endoscope is rigid.

Embodiment 29: The load sensing device of embodiment 27, wherein the endoscope is flexible.

Embodiment 30: The load sensing device of any one of embodiments 22-29, further comprising indicators to alert the users visually, audibly, tactilely, or a combination thereof as the input force increases.

Embodiment 31: The load sensing device of embodiment 30, wherein the indicators are visual indicators comprising a green visual indicator, a yellow visual indicator, a red visual indicator, a black visual indicator, or a combination thereof.

Embodiment 32: The load sensing device of embodiment 30, wherein the indicators are visual indicators, comprising flashing green, yellow, and red lights.

Embodiment 33: The load sensing device of embodiment 30, wherein the indicators are an audible indicator, comprising increasing and/or disharmonious sounds as force increases.

Embodiment 34: The load sensing device of any one of embodiments 22-33, wherein the load sensing device is not attached to the UAS but instead moves freely along the guidewire.

Embodiment 35: The load sensing device of any one of embodiments 22-34, wherein the force detecting, and force output is done continuously.

Embodiment 36: The load sensing device of any one of embodiments 22-35, further comprising an external computer with software adapted to continuously collect and store the force value.

Embodiment 37: The load sensing device of embodiment 36, wherein the force value collection and storage is done wirelessly.

Embodiment 38: The load sensing device of embodiment 37, wherein the collection and storage is done by ultra-high frequency radio waves limited in the 2.4 GHz band for wireless data transmission technology.

Embodiment 39: The load sensing device of any one of embodiments 22-38, wherein the adjustable attachment mechanism is a screw port.

Embodiment 40: The load sensing device of any one of embodiments 22-39, wherein the adjustable attachment mechanism is a mechanical screw port.

Embodiment 41: The load sensing device of any one of embodiments 22-39, wherein the adjustable attachment mechanism is a magnetic screw port.

Embodiment 42: The load sensing device of any one of embodiments 22-41, wherein the surgical device is a device for an endoscopic, laparoscopic, robotic, and/or minimally invasive surgical procedure.

Embodiment 43: The load sensing device of any one of embodiments 22-42, wherein the input/output interface is used in a disposable arrangement.

Embodiment 44: The load sensing device of any one of embodiments 22-43, wherein a user is capable of continuously measuring input force in real time during the deployment of the surgical device in a patient.

What is claimed is:

1. A handheld load sensing device, comprising:
a) a cylindrical outer casing (100), wherein the cylindrical outer casing (100) comprises a first casing end (110), a second casing end (120), an exterior lumen (131), and a guidewire lumen (132) disposed within the exterior lumen (131), wherein the first casing end (110) comprises a first opening (111) and the second casing end (120) comprises a second opening (122) for accessing the guidewire lumen (132);
b) a plunger (200) slidably coupled to the cylindrical outer casing (100), wherein the plunger (200) comprises a first plunger end (210) and a second plunger end (220) having a handle (300) disposed thereon, wherein the first plunger end (210) is disposed through the first opening (111) of the first casing end (110) and within the exterior lumen (131), wherein the first plunger end (210) comprises a plunger lumen (230), wherein the plunger lumen (230) and the guidewire lumen (132) of the cylindrical outer casing (100) are fluidly coupled; and
c) a spring (400) disposed within the exterior lumen (131) of the cylindrical outer casing (100), wherein a first spring end (410) of the spring rest against the second casing end (120) and a second spring end (420) rest against the first plunger end (210), wherein the guidewire lumen (132) is disposed through the spring (400) such that the spring (400) wraps around the guidewire lumen (132); wherein when the handle (300) of the plunger (200) is pushed towards the first casing end (110), the plunger (200) pushes against and compresses the spring (400), wherein displacement of the spring (400) corresponds to an applied force.

2. The device of claim 1, wherein the second casing end (120) of the cylindrical outer casing (100) further comprises an interface for a ureteral access sheath (UAS) or an endoscope.

3. The device of claim 1, wherein the interface comprises a Luer-Lock style interface.

4. The device of claim 2, wherein the interface is positioned distal to UAS point of insertion.

5. The device of claim 1, further comprising a locking mechanism.

6. The device of claim 5, wherein the locking mechanism is a pawl and ratchet mechanism.

7. The device of claim 6, wherein the pawl and ratchet mechanism is engaged by twisting the handle (300) counterclockwise.

8. The device of claim 6, wherein the pawl and ratchet mechanism is disengaged by twisting the handle (300) clockwise.

9. The device of claim 1, wherein the outer cylindrical casing (100) further comprises an indicator window.

10. The device of claim 9, wherein the indicator window displays visual indicators corresponding to a force being applied.

11. The device of claim 10, wherein the visual indicators comprise a green visual indicator, a yellow visual indicator, a red visual indicator, a black visual indicator, or a combination thereof.

12. A handheld load sensing device, comprising
a) a force sensor to receive a force;
b) an input/output interface, and
c) an adjustable attachment mechanism adapted to reversibly attach to a surgical device which is passed over a guidewire;
wherein the force sensor detects the amount of force applied to the butt end of a catheter during deployment of the surgical device over the guidewire in a patient and outputs a force value representative thereof through the input/output interface.

13. The load sensing device of claim 12, wherein the force sensor is mechanical.

14. The load sensing device of claim 13, wherein the force sensor is a spring.

15. The load sensing device of claim 12, wherein the force sensor is electromechanical.

16. The load sensing device of claim 12, wherein the force sensor is a load cell.

17. The load sensing device of claim 12, wherein the surgical device is a ureteral access sheath (UAS) or an endoscope.

18. The load sensing device of claim 12, further comprising indicators to alert the users visually, audibly, tactilely, or a combination thereof as the input force increases.

19. The load sensing device of claim 12, wherein the force detecting, and force output is done continuously.

20. The load sensing device of claim 12, wherein the adjustable attachment mechanism is a screw port.
